(19) United States
(12) Patent Application Publication
(10) Pub. No.: US 2022/0265667 A1
(43) Pub. Date: Aug. 25, 2022

(54) ETIFOXINE FOR USE IN THE TREATMENT OF DISEASES RELATED TO ACTIVATED MAST CELLS

(71) Applicant: MC Sciences UG, Meckenheim (DE)

(72) Inventor: Gerhard J. MOLDERINGS, Meckenheim (DE)

(73) Assignee: MC Sciences UG, Meckenheim (DE)

(21) Appl. No.: 17/632,592

(22) PCT Filed: Aug. 6, 2020
(86) PCT No.: PCT/EP2020/072100

§ 371 Date: Feb. 3, 2022

(30) Foreign Application Priority Data
Aug. 6, 2019 (EP) 19190289.9

(51) Int. Cl. A61K 31/536

(52) U.S. Cl. CPC A61K 31/536

(57) ABSTRACT

Etifoxine, or a pharmaceutically acceptable derivative thereof, can be used for the treatment of a disease related to activated mast cells in a subject, preferably a human. Furthermore, a pharmaceutical composition containing Etifoxine, or a pharmaceutically acceptable derivative thereof, and a pharmaceutically acceptable excipient can be used for the treatment of a disease related to activated mast cells. An in vitro or ex-vivo method of inhibiting mast cell activation, involves contacting a cell with Etifoxine, or a pharmaceutically acceptable derivative thereof.

### BACKGROUND OF THE INVENTION

### Field of the Invention

The present disclosure relates to Etifoxine, or a pharmaceutically acceptable derivative thereof, for use in the treatment of a disease in which activated mast cells are involved. The present disclosure also relates to pharmaceutical compositions comprising Etifoxine, or a pharmaceutically acceptable derivative thereof, and a pharmaceutically acceptable excipient for use in the treatment of a disease related to activated mast cells. Further, the present disclosure provides methods of inhibiting mast cell activation with Etifoxine, or a pharmaceutically acceptable derivative thereof.

### Background of the Invention

Mast cells are hematopoietic tissue immune cells that secrete pre-stored mediators, such as histamine and tryptase, as well as, numerous de novo synthesized chemokines and cytokines in response to allergic or non-immune triggers. Mast cells act both as effector cells as well as regulatory cells and play central roles in adaptive and innate immunity. This versatility is reflected in numerous activation stimuli with intracellular pathways that intersect to modulate the quality and magnitude of the mast cell response. The best-characterized mechanism of mast cell activation is antigen-mediated cross-linking of IgE bound to FcεRI on mast cells. In addition, a large number of IgE-independent modes of mast cell activation have been described. Human mast cells express a multiplicity of G-protein-coupled receptors and other recognition sites on their surface which are involved in mast cell activation under physiological and pathophysiological conditions. Stimulation of these receptors can either result in potentiation of FcεRI-mediated mast cell activation or induction of mast cell activation by themselves using different intracellular complementary, as well as, converging signaling pathways.

Apart from being prominently involved in allergic reactions, mast cells are critical for the maintenance of tissue integrity and function. This correlates with their ubiquitous presence in almost all tissues. Their central role in immunological as well as non-immunological processes is also reflected by the large number of mediators (over 200 different mediators known to date) by which mast cells may influence other cells. The profile of mediators stored or produced de novo in mast cells can markedly differ between and within organs/tissues, depending on the microenvironmental factors or the nature of the stimulus.

Since mast cells are located at the border of the body and environment, they are perfectly equipped with their mediators to orchestrate the immune system. They can recruit other immune cells to the site of injury and control the function of various cells, such as eosinophilic granulocytes, T and B lymphocytes, thereby being implicated in the protection of the organism against bacterial, parasitic and viral infections. This role can be achieved precisely because mast cells are able both to degranulate and to selectively release specific mediators by differential release.

In addition, mast cells essentially regulate homeostasis, e.g. by contributing to wound healing as well as tissue remodeling (e.g., in hair follicles and bones). Mast cells promote homeostasis also by degrading certain endogenous toxins such as endothelin-1 or neurotensin released in response to bacterial infection by means of their potent proteases. Similarly, mast cells are involved in the control of exogenous toxins, such as venoms and bacterial toxins.

The very same features that enable mast cells to protect the organism can wreak havoc to the organism when running out of control. Mast cells are known to be the primary responders in allergic reactions, orchestrating strong responses to minute amounts of allergens. The broad spectrum of functions of mast cells might explain why mast cells can be involved in so many different pathologies beyond allergy (see Modena, “Emerging concepts: mast cell involvement in allergic diseases”, Translational Research 174: 98-121 (2016)) such as asthma/COPD, atherosclerosis (see Kovanen, “Mast cells in atherosclerotic cardiovascular disease—Activators and actions”, European Journal of Pharmacology 816: 37-46 (2017)), autoimmune diseases, atopic dermatitis, cardiac arrhythmias, chronic fatigue syndrome (see Hatziagelaki, “Myalgic encephalomyelitis/chronic fatigue syndrome—Metabolic disease or disturbed homeostasis due to focal inflammation in the hypothalamus?”, The Journal of Pharmacology and Experimental Therapeutics 367: 155-167 (2018)), dengue fever (see Londono-Renteria, “Role of Mast Cells in Dengue Virus Pathogenesis”, DNA and Cell Biology 36: 423-427 (2017)), eosinophilic esophagitis, eosinophilic gastritis, eosinophilic enteritis and colitis, fibromyalgia (see Irene Tsilioni, Irwin J. Russell, Julia M. Stewart, Rae M. Gleason, and Theoharis C. Theoharides, Neuropeptides CRH, SP, HK-1, and Inflammatory Cytokines IL-6 and TNF Are Increased in Serum of Patients with Fibromyalgia Syndrome, Implicating Mast Cells, J Pharmacol Exp Ther 356:664-672, March 2016), fibrotic diseases, heart failure, irritable bowel syndrome (see T. Frieling, K. Meis, U. W. Kolck, J. Homann, A. Hülsdonk, U. Haars, H.-J. Hertfelder, J. Oldenburg, H. Seidel, G. J. Molderings, Evidence for mast cell activation in patients with therapy-resistant irritable bowel syndrome, Z Gastroenterol 2011; 49: 191-194), inflammatory bowel disease, interstitial cystitis (see Regauer, “Mast cell activation syndrome in pain syndromes bladder pain syndrome/interstitial cystitis and vulvodynia”, Translational Anthology and Urology 5: 396-397 (2016)), systemic mastocytosis, systemic mast cell activation syndrome (seeAfrin, B. Lawrence, Mast cell activation disease and the modern epidemic of chronic inflammatory disease, Translational Research, Volume 174, August 2016, Pages 33-59), mast cell leukaemia, migraines, neurofibromatosis, non-cardiac chest pain, osteoporosis, peritoneal adhesions, rheumatoid arthritis, rosacea, sarcoidosis, thrombosis and vulvodynia (see Regauer, “Mast cell activation syndrome in pain syndromes bladder pain syndrome/interstitial cystitis and vulvodynia”, Translational Andrology and Urology 5: 396-397 (2016)).

Activated mast cells are being wildly explored as a highly attractive biological target for the treatment of a plethora of diseases comprising but not limited to the following:

Mast cell degranulation can influence the pathology of allergic disorders and can either directly affect structural cells residing in the affected tissues or influence the pathology indirectly through effects of mast cells on dendritic cells, T cells, B cells and other hematopoietic cell types (see Modena B. D., Dazy K., White A. A.: Emerging concepts: mast cell involvement in allergic diseases, Translational Research 2016 August; 174:98-121).

Activated mast cells have detrimental effects on the vessel wall and the atherosclerotic plaque by the release of mast cell specific proteases such as chymase and tryptase, as well as cytokines, chemokines and growth factors. Activated mast cells increase atherosclerotic lesion areas, as well as plasma total cholesterol, LDL, and triglyceride levels. The concerted actions of arterial mast cells have the potential to contribute to the initiation and progression of atherosclerosis, and ultimately to destabilization and rupture of an advanced atherosclerotic plaque with ensuing atherothrombotic complications (see Petri T. Kovanena, Ilze Bot. 2017. Mast cells in atherosclerotic cardiovascular disease—Activators and actions. European Journal of Pharmacology Volume 816, 5 Dec. 2017. Pages 37-46).

Human clinical studies demonstrate a significant causal involvement of activated mast cells in Interstitial Cystitis (IC) and Bladder Pain Syndrome (BPS). An increased sub-epithelial mast cell count was seen, in particular in IC with Hunner lesions biomarker with close apposition of mast cells to nerve fibers in the urinary bladder (see Sigrid Regauer. 2016. Mast cell activation syndrome in pain syndromes bladder pain syndrome/interstitial cystitis and vulvodynia. Transl Androl Urol 2016. 5(3):396-397).

Chronic Fatigue Syndrome (CFS)/Myalgic Encephalomyelitis (ME) is quite common in patients with systemic mast cell activation. Peptides secreted by inflammatory, immunologic stimulation, physical or emotional acute stress activate diencephalic mast cells, either directly or through neuropeptides. Further, mast cells could stimulate the hypothalamic-pituitary-adrenal axis. A study has revealed increased activated mast cells in CFS/ME patients compared with healthy controls (see Hatziagelaki E., Adamaki M., Tsilioni I., Dimitriadis G., Theoharides T C.: Myalgic Encephalomyelitis/Chronic Fatigue Syndrome-Metabolic Disease or Disturbed Homeostasis due to Focal Inflammation in the Hypothalamus?, J Pharmacol Exp Ther. 2018 October; 367(1):155-167).

Chronic lyme disease can be disguised as mast cell activation disease, wherein mast cells are activated as the healthy immune response to the lyme disease infection, for example by Borrelia burgdorferi. In some cases, although the lyme disease inducing bacteria have been depleted and the actual initiating trigger has been removed, still the lyme disease symptoms prevail, potentially caused by still chronically activated mast cells in tissues, organs and brain (Allison DeLong, Mayla Hsu, Harriet Kotsoris: Estimation of cumulative number of posttreatment Lyme disease cases in the US, 2016 and 2020, BMC Public Healthvolume 19, Article number: 352 (2019).

Furthermore, scientific and medical evidence has shown a crucial role of mast cell activation in thrombotic diseases such as deep vein thrombosis or venous thromboembolism. It was demonstrated that mast cells likely exert their pro-thrombotic actions through release of granular constituents including histamine, which possibly leads to endothelial activation and Weibel-Palade body release, platelet adhesion, and further leukocyte recruitment (Alex L. Huang, Julian J. Bosco, Karlheinz Peter. 2017. Mast Cell—An unexpected villain in venous thromboembolism? Circ Res. 2017.121:899-901).

Dengue virus infection is the cause of the debilitating diseases dengue fever and dengue hemorrhagic fever. Mast cells are strongly activated by the Dengue virus which results in activation and degranulation of potent vasoactive mediators pre-stored in the granules that act directly on the vascular endothelium and induce vascular leakage. Thereby, the mast cell activation levels correlate with disease severity in human patients. (see Berlin Londono-Renteria, Julio C. Marinez-Angarita, Andrea Troupin, and Tonya M. Colpitts: Role of Mast Cells in Dengue Virus Pathogenesis, DNA and Cell Biology 2017 36:6, 423-427)

Fibromyalgia is a chronic, idiopathic condition of widespread musculoskeletal pain that is clinically characterized by muscle and soft tissue aches and tenderness, chronic fatigue, anxieties, sleep disturbances, as well as, cognitive dysfunction. Fibromyalgia is common in patients with systemic mast cell activation disease leading to blood-brain-barrier disruption through brain mast cell activation (see Irene Tsilioni, Irwin J. Russell, Julia M. Stewart, Rae M. Gleason, and Theoharis C. Theoharides, Neuropeptides CRH, SP, HK-1, and Inflammatory Cytokines IL-6 and TNF Are Increased in Serum of Patients with Fibromyalgia Syndrome, Implicating Mast Cells, J Pharmacol Exp Ther 356:664-672, March 2016)

Mast cells with their pivotal role in tissue repair and remodeling are crucially involved in the pathogenesis of fibrotic diseases, such as idiopathic pulmonary fibrosis, cardiac fibrosis and renal fibrosis, with often high morbidity rate. In patients with pulmonary fibrosis, the mast cell density is significantly increased (up to 10 times higher than in normal patients) correlating with the degree of fibrosis, lung function and clinical disease progression (see Shimbori C., Upagupta C., Forsythe P., Kolb M.: The Role of Mast Cells in the Pathophysiology of Pulmonary Fibrosis. In: Willis M., Yates C., Schisler J. (eds) Fibrosis in Disease. Molecular and Translational Medicine. Humana Press, Cham, 2019).

In addition to that, mast cells play a significant role in several aspects of Colitis Ulcerosa, including regulation of epithelium permeability, transmittance of signals during neuropathologic stress, and inflammatory responses with the subsequent tissue remodeling including fistula and strictures. The release of pro-inflammatory mediators from activated mast cells in the intestine and colon of patients contributes to the inability of the epithelium to act as an effective barrier to pathogens and immune-activating antigens in the gastrointestinal tract. Such patients often show an increased number of mast cells in close apposition to the basal lamina of the glands among the epithelial cells and in other layers of the gut wall (see Matthew J. Hamilton, MD, Sandra M. Frei, and Richard L. Stevens, PhD, The Multifaceted Mast Cell in Inflammatory Bowel Disease, Inflamm Bowel Dis. 2014 December; 20(12): 2364-2378.)

Mast cell hyperactivation, furthermore, plays a crucial role in the pathophysiology of Irritable Bowel Syndrome. The abnormally activated mast cells are directly related to visceral hypersensitivity, abdominal pain, diarrhea frequency and further symptoms, thus the activation of mast cells participates in multiple pathophysiological processes of Irritable Bowel Syndrome (see T. Frieling, K. Meis, U. W. Kolck, J. Homann, A. Hülsdonk, U. Haars, H.-J. Hertfelder, J. Oldenburg, H. Seidel, G. J. Molderings, Evidence for mast cell activation in patients with therapy-resistant irritable bowel syndrome, Z Gastroenterol 2011; 49: 191-194).

Mast cell activation disease includes mast cell activation syndrome, as well as the rare disease variant systemic mastocytosis, featuring inappropriate mast cell activation leading to widespread systemic symptoms of an inflammatory or allergic theme (seeAfrin, B. Lawrence, Mast cell activation disease and the modern epidemic of chronic inflammatory disease, Translational Research, Volume 174, August 2016, Pages 33-59).

Systemic lupus erythematosus is a complex chronic inflammatory autoimmune disease that can cause multiple tissue and organ damages such as arthritis, skin lesions, hematologic changes, renal and neurologic disorders. Among various immune cells that mediate inflammation in systemic lupus erythematosus, it has been increasingly shown that mast cell activation plays a decisive role to the disease manifestation (see Al. Caraffa et al. 2019. Impact of mast cells in systemic lupus erythematosus: can inflammation be inhibited? Journal of Biological regulators & homeostatic agents. Vol. 33, no. 3, 669-673 (2019)).

In addition to that, obesity is caused by abnormal mast cell activity in at least some portion of the obese population. White adipose tissue has been identified as a reservoir of mast cell progenitors resulting in enhanced neo-vascularization and macrophage accumulation. Mast cells also affect adipose tissue remodelling and fibrosis by adipocyte differentiation and fibroblast proliferation. In clinical practice, patients with activated mast cells often have elevations in total cholesterol and low-density lipoproteins and decreases in high-density lipoproteins and hypertriglyceridemia (see Brian S. Finlin, Beibei Zhu. 2017. Mast Cells Promote Seasonal White Adipose Beiging in Humans. Diabetes 2017; 66:1237-1246).

Also Chronic Spontaneous Urticaria or inducible urticaria are caused by the inappropriate activation and degranulation of dermal mast cells. Increased numbers of mast cells are found in both lesional and non-lesional skin in Chronic Spontaneous Urticaria and inducible urticaria. Various activating factors stimulate mast cells to secrete vasoactive molecules that activate endothelial cells leading to increased vasopermeability and extravascular leakage of fluids and proteins (urticarial wheals). Mast cell degranulation in the area of wheals has been demonstrated repeatedly (see Jain S.: Pathogenesis of Chronic Urticaria: An Overview, Dermatology Research and Practice 2014; 2014:674709).

Mast cells are substantially involved in viral infections, such as SARS-CoV-2 (Covid-19), promoting irreversible fibrotic lung damages. After first contact with the virus, mast cells release cytokines and mediators to defend against the viral infection and support the important clearing of the viral infection. In some cases, however, the human immune system is not able to clear the viral infection and the immune system moves into a stage of hyper-inflammation, in some cases due to medical pre-conditions of the patient. In the stage of hyper-inflammation, the mast cells play a central role and are significantly involved in the life-threatening cytokine storm, resulting in possible sepsis with fatal organ damage (see Zhuo Zhou, Lili Ren et al. 2020. Heightened innate immune responses 1 in the respiratory tract of SARS-CoV-2 (Covid-19) patients. Cell Press. DOI: 10.1016/j.chom.2020.04.017; K. Kritas, G. Ronconi, Al. Caraffa, C. E. Gallenga, R. Ross, P Conti. 2020. Mast Cells Contribute to Coronavirus-Induced Inflammation: New Anti-Inflammatory Strategy. Biolife Sas. PMID: 32013309, DOI: 10.23812/20-Editorial-Kritas).

Numerous other highly pathological viral infections, such as acute respiratory disease syndrome (ARDS), influenza A, SARS-CoV-2 (Covid-19), SARS-CoV, MERS-CoV, and hemorrhagic viruses such as Marburg and Ebola, also cause significant disease through immune-mediated pathology to tissue and/or induction of vascular permeability. In addition to macrophages and neutrophils, mast cells contribute to an excessive inflammatory response and vascular problems (see Amy C. Graham, Rachel M. Temple and Joshua J. Obar. 2015. Mast cells and influenza A virus: association with allergic responses and beyond. Frontiers in Immunology. May 2015, Volume 6, Article 238).

There is still a need in the art for drugs useful for treating disorders related to activated mast cells. There is further the need of effective and selective drugs for targeting activated mast cells and attenuate the degree of activation of the mast cells. There is also the need of easily available drugs useful for treating disorders related to activated mast cells. There is also the need of well-tested drugs useful for treating disorders related to activated mast cells.

### SUMMARY OF THE INVENTION

It is thus one object of the present disclosure to provide an alternative treatment of diseases related to activated mast cells.

In one aspect, the present disclosure provides Etifoxine, or a pharmaceutically acceptable derivative thereof, for use in the treatment of a disease related to activated mast cells in a subject, preferably a human.

In a second aspect, the present disclosure provides a pharmaceutical composition comprising Etifoxine, or a pharmaceutically acceptable derivative thereof, and a pharmaceutically acceptable excipient for use in the treatment of a disease related to activated mast cells.

In a third aspect, the present disclosure provides an in vitro or ex vivo method of inhibiting mast cell activation, comprising contacting the mast cell with Etifoxine, or a pharmaceutically acceptable derivative thereof.

### BRIEF DESCRIPTION OF THE FIGURES

FIG. 1. Dose-dependent in vitro release of β-hexosaminidase (“% degranulation”) of unstimulated (unst) and stimulated (Ag) murine mast cells, respectively, in the presence of increasing doses of DMSO (for comparative purpose) and Etifoxine.

FIG. 2. Dose-dependent in vitro release of Interleukin-6 (IL-6) of unstimulated (unst) and stimulated (Ag) murine mast cells, respectively, in the presence of increasing doses of DMSO (for comparative purpose) and Etifoxine.

FIG. 3. Time-dependent ex vivo release of tryptase of unstimulated (without stimulation; diamond labeling) and LPS-stimulated (square, star and circular labeling) human nasal tissue mast cells in the absence (DMSO 0) and presence of DMSO (DMSO 1:450; DMSO 1:900). DMSO 0 means the ratio DMSO:media=0:1. DMSO 1:450 means the ratio DMSO:media=1:450. DMSO 1:900 means the ratio DMSO:media=1:900. For comparative purposes.

FIG. 4. Time-dependent ex vivo release of tryptase of unstimulated (without stimulation; diamond labeling) and LPS-stimulated (square, star, circular, vertical line and no labeling) human nasal tissue mast cells in the absence of Etifoxine (0 μM) and presence of different Etifoxine concentrations (60 μM, 30 μM, 10 μM, 3 μM).

FIG. 5. Dose-dependent ex vivo release of tryptase upon LPS stimulation after two hours and four hours, respectively, in the presence of Etifoxine. Concentrations of Etifoxine (3 μM, 10 μM, 30 μM, 60 μM; from left to right) are presented as the respective logarithmic values. For the purpose of IC₅₀ determination.

FIG. 6. Dose-dependent ex vivo release of tryptase upon SCF stimulation after four hours and eight hours, respectively, in the presence of Etifoxine. Concentrations of Etifoxine (1 μM, 3 μM, 10 μM, 30 μM, 60 μM, 150 μM, 300 μM; from left to right) are presented as the respective logarithmic values. For the purpose of IC₅₀ determination.

FIG. 7. Time-dependent ex vivo release of tryptase of unstimulated (without stimulation; diamond labeling) and SCF-stimulated (square, triangle, x, star, circular, vertical line, no labeling and dashed line) human nasal tissue mast cells in the absence of Etifoxine (0 μM) and presence of different Etifoxine concentrations (300 μM, 150 μM, 60 μM, 30 μM, 10 μM, 3 μM, 1 μM).

FIG. 8. Total clinical symptom intensity score in VAS during the 6 week treatment with Etifoxine (Off-label use) in a patient with systemic mastocytosis.

FIG. 9. Clinical symptom intensity score in VAS for each clinical symptom category during the 6 week treatment with Etifoxine in a patient with systemic mastocytosis

### DETAILED DESCRIPTION OF THE INVENTION

Hereinafter, the aspects of the present disclosure will be described in detail by means of embodiments.

The present disclosure as illustratively described in the following may suitably be practiced in the absence of any element or elements, limitation or limitations not specifically disclosed herein.

The present disclosure will be described with respect to particular embodiments and with reference to certain figures, but the disclosure is not limited thereto, but only by the claims. Terms as set forth hereinafter are generally to be understood in their common sense unless indicated otherwise.

### Definitions

Where the term “comprising” is used in the present description and claims, it does not exclude other elements. For the purposes of the present disclosure, the term “consisting of” is considered to be a preferred embodiment of the term “comprising”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the”, this includes a plural of that noun unless specifically stated otherwise.

The term “about,” as used herein in connection with a measured quantity, refers to the normal variations in that measured quantity, as expected by the skilled artisan making the measurement and exercising a level of care commensurate with the objective of measurement and the precision of the measuring equipment. Typically, the term “about” includes the recited number±10%. Thus, “about 10” means 9 to 11.

Terms like “obtainable” and “obtained” are used interchangeably. This, e.g., means that, unless the context clearly dictates otherwise, the term “obtained” does not mean to indicate that e.g. an embodiment must be obtained by e.g. the sequence of steps following the term “obtained” even though such a limited understanding is always included by the terms “obtained” as a preferred embodiment.

In the present disclosure, the term “S-Etifoxine” relates to the S-enantiomer in its substantially pure form and it is substantially free from R-Etifoxine (i.e., in enantiomeric excess). As used herein, “S-Etifoxine” denotes that the compound comprises more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer S-Etifoxine.

As used herein and unless otherwise indicated, the term “S-Etifoxine” refers to at least about 90% by weight S-Etifoxine and at most about 10% by weight R-Etifoxine, at least about 95% by weight S-Etifoxine and at most about 5% by weight R-Etifoxine, at least about 99% by weight S-Etifoxine and at most about 1% by weight R-Etifoxine or at least about 99.9% by weight S-Etifoxine and at most about 0.1% by weight R-Etifoxine.

As used herein and unless otherwise indicated, the term the term “R-Etifoxine” relates to the R-enantiomer in its substantially pure form and it is substantially free from S-Etifoxine (i.e., in enantiomeric excess). As used herein, “R-Etifoxine” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer R-Etifoxine.

As used herein and unless otherwise indicated, the term “R-Etifoxine” refers to at least about 80% by weight R-Etifoxine and at most about 20% by weight S-Etifoxine, at least about 90% by weight R-Etifoxine and at most about 10% by weight S-Etifoxine, at least about 95% by weight R-Etifoxine and at most about 5% by weight S-Etifoxine, at least about 99% by weight R-Etifoxine and at most about 1% by weight S-Etifoxine, at least about 99.9% by weight R-Etifoxine or at most about 0.1% by weight S-Etifoxine.

The term “enantiomer” or “enantiomeric” refers to a molecule that is nonsuperimposeable on its mirror image and hence optically active where the enantiomer rotates the plane of polarized light in one direction and its mirror image rotates the plane of polarized light in the opposite direction.

The terms “racemate” or “racemic” refer to an optically inactive mixture of equal parts of enantiomers. As used herein, the term “racemic Etifoxine” refers to an optically inactive mixture of equal parts of S- and R-Etifoxine.

As used herein, the terms “IC₅₀” or IC₅₀ value” refers to an amount, concentration or dosage of a particular test compound that achieves a 50% inhibition of a maximal response in an assay that measures such response.

The terms “co-administration” or “in combination with” include the administration of two therapeutic agents (e.g., Etifoxine and docetaxel) either simultaneously, concurrently or sequentially with no specific time limits. In one embodiment, both agents are present in a subject at the same time or exert their biological or therapeutic effect at the same time. In one embodiment, the two therapeutic agents are in the same composition or unit dosage form. In another embodiment, the two therapeutic agents are in separate compositions or unit dosage forms.

The phrase “pharmaceutically acceptable derivative”, as used herein, includes any pharmaceutically acceptable salt, solvate, radiolabeled, stereoisomer, enantiomer, other stereoisomeric form, racemic mixture, geometric isomer, and/or tautomer of Etifoxine. In one embodiment, the pharmaceutically acceptable derivative is a pharmaceutically acceptable salt, solvate, radiolabeled, stereoisomer, enantiomer, other stereoisomeric form, racemic mixture, geometric isomer, and/or tautomer of Etifoxine. In another embodiment, the pharmaceutically acceptable derivative is a pharmaceutically acceptable salt of Etifoxine.

The phrase “pharmaceutically acceptable salt”, as used herein, is any pharmaceutically acceptable salt that can be prepared from Etifoxine including a salt formed from an acid and a basic functional group, such as a nitrogen group of Etifoxine. Illustrative salts include, but are not limited, to sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

As used herein, the terms “stereoisomer”, “stereoisomeric form”, and the like are general terms of all isomers of individual molecules that differ only in the orientation of their atoms in space (comprising enantiomers).

“Solvates” are known in the art and are considered to be a combination, physical association and/or solvation of Etifoxine with a solvent molecule, e.g., a disolvate, monosolvate or hemisolvate when the ratio of the solvent molecule to the molecule of Etifoxine is 2:1, 1:1 or 1:2, respectively. This physical association involves varying degrees of ionic and covalent bonding, including hydrogen bonding. In certain instances, the solvate can be isolated, for example, when one or more solvent molecules are incorporated into the crystal lattice of a crystalline solid. Thus, “solvate”, as used herein, encompasses both solution-phase and isolatable solvates. Etifoxine may be present as a solvated form with a pharmaceutically acceptable solvent, such as water, methanol, ethanol, and the like, and it is intended that the disclosure include both solvated and unsolvated Etifoxine forms. As “hydrate” relates to a particular subgroup of solvates, i.e., where the solvent molecule is water, hydrates are included within the solvates of the disclosure. Preparation of solvates is known in the art. For example, M. Caira et al., J. Pharmaceut. Sci., 93(3):601-611 (2004), describes the preparation of solvates of fluconazole with ethyl acetate and with water. Similar preparations of solvates, hemisolvate, hydrates, and the like are described by E. C. van Tonder et al., AAPS Pharm. Sci. Tech., 5(1), article 12 (2004), and A. L. Bingham et al., Chem. Commun., 603-604 (2001). A typical, non-limiting, process involves dissolving Etifoxine in a desired amount of the desired solvent (organic, water or mixtures thereof) at temperatures above about 20° C. to about 25° C., cooling the solution at a rate sufficient to form crystals, and isolating the crystals by known methods, e.g., filtration. Analytical techniques, for example, infrared spectroscopy, can be used to show the presence of the solvent in a crystal of the solvate.

In addition, one or more hydrogen, carbon or other atoms of Etifoxine can be replaced by a radioactive isotope of the hydrogen, carbon or other atoms. Such a “radiolabeled”, “radiolabeled form”, and the like of Etifoxine is useful as a research and/or diagnostic tool in metabolism pharmacokinetic studies and in binding assays. “Radioactive”, as used herein with respect to an atom, means an atom that comprises a radioactive atom and therefore the specific radioactivity thereof is above the background level of radioactivity. Examples of radioactive isotopes that can be incorporated into Etifoxine include isotopes of hydrogen, carbon, nitrogen, oxygen, phosphorous, sulfur, fluorine, chlorine, bromine, and iodine, such as ²H, ³H, ¹¹C, ¹³C, ¹⁴C, ¹⁵N, ¹⁷O, ¹⁸O, ³¹P, ³²P, ³⁵S, ¹⁸F, ¹⁹F, ³⁶Cl, ³⁷Cl, ⁷⁶Br, ⁷⁷Br, ⁸¹Br, ¹²³I, ¹²⁴I, ¹²⁵I, and ¹³¹I, respectively. In one embodiment, radiolabeled Etifoxine contains 1, 2, 3, 4, or more radioactive isotopes, each of which is independently selected from hydrogen, carbon, nitrogen, oxygen, phosphorous, sulfur, fluorine, chlorine, bromine, and iodine. In another embodiment, radiolabeled Etifoxine contains 1 or 2 radioactive isotopes, each of which is independently selected from hydrogen, carbon, nitrogen, oxygen, phosphorous, sulfur, fluorine, chlorine, bromine, and iodine. In another embodiment, radiolabeled Etifoxine contains 1 (one) radioactive isotope which is selected from hydrogen, carbon, nitrogen, oxygen, phosphorous, sulfur, fluorine, chlorine, bromine, and iodine. In another embodiment, radiolabeled Etifoxine contains 1, 2, 3, 4, or more radioactive isotopes, each of which is independently selected from ²H, ³H, ¹¹C, ¹³C, ¹⁴C, ¹⁵N, ¹⁷O, ¹⁸O, ³¹P, ³²P, ³⁵S, ¹⁸F, ¹⁹F, ³⁶Cl, ³⁷Cl, ⁷⁶Br, ⁷⁷Br, ⁸¹Br, ¹²³I, ¹²⁴I, ¹²⁵I, and ¹³¹I. In another embodiment, adiolabeled Etifoxine contains 1 or 2 radioactive isotopes, each of which is independently selected from ²H, ³H, ¹¹C, ¹³C, ¹⁴C, ¹⁵N, ¹⁷O, ¹⁸O, ³¹P, ³²P, ³⁵S, ¹⁸F, ¹⁹F, ³⁶Cl, ³⁷Cl, ⁷⁶Br, ⁷⁷Br, ⁸¹Br, ¹²³I, ¹²⁴I, ¹²⁵I, and ¹³¹I. In another embodiment, radiolabeled Etifoxine contains 1 radioactive isotope which is selected from ²H, ³H, ¹¹C, ¹³C, ¹⁴C, ¹⁵N, ¹⁷O, ¹⁸O, ³¹P, ³²P, ³⁵S, ¹⁸F, ¹⁹F, ³⁶Cl, ³⁷Cl, ⁷⁶Br, ⁷⁷Br, ⁸¹Br, ¹²³I, ¹²⁴I, ¹²⁵I, and ¹³¹I. In another embodiment, radiolabeled Etifoxine contains 1, 2, 3, 4, or more radioactive isotopes, each of which is independently selected from ²H, ³H, ¹³C, ¹⁴C, ¹⁵N, ¹⁸O, ³²P, and ¹²⁵I. In another embodiment, radiolabeled Etifoxine contains 1 or 2 radioactive isotopes, each of which is independently selected from ³H, ¹⁴C, ¹⁵N, ¹⁸O, ³²P, and ¹²⁵I. In another embodiment, radiolabeled Etifoxine contains 1 (one) radioactive isotope which is selected from ³H, ¹⁴C, ¹⁵N, ¹⁸O, ³²P, and ¹²⁵I. In another embodiment, radiolabeled Etifoxine contains 1 (one) Deuterium (²H) atom.

Radiolabeled Etifoxine can be prepared by methods known in the art. For example, tritiated Etifoxine can be prepared by introducing tritium into Etifoxine, for example, by catalytic dehalogenation with tritium. This method can include reacting a suitably halogen-substituted precursor of Etifoxine with tritium gas in the presence of a suitable catalyst, for example, Pd/C, in the presence or absence of a base. Other suitable methods for preparing tritiated Etifoxine can be found in Filer, “The Preparation and Characterization of Tritiated Neurochemicals,” Chapter 6, pp. 155-192 in Isotopes in the Physical and Biomedical Sciences, Vol. 1, Labeled Compounds (Part A) (1987). ¹⁴C-labeled Etifoxine can be prepared by employing starting materials having a ¹⁴C carbon.

The term “pharmaceutically acceptable excipient” as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each excipient must be “acceptable” in the sense of being compatible with the other ingredients of the formulation and not injurious to the patient.

Pharmaceutically acceptable excipients include, but are not limited thereto, binder, filler, diluent, disintegrating agent, lubricant, glidant, flavouring agent, wetting agent, emulsifying agent, pH buffering agent, sweetening agent, and a mixture thereof. Some examples of materials which can serve as pharmaceutically acceptable excipients are sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, marmitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, binders, glidants, disintegrating agents, pH buffering agents, release agents, coating agents, sweetening, flavoring and perfuming agents, preservatives and antioxidants can also be present in the compositions.

As used herein, “binders” suitable for use in pharmaceutical compositions include, but are not limited thereto, corn, starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose, calcium carboxymethyl cellulose, sodium carboxymethyl cellulose), polyvinyl pyrrolidone, methyl cellulose, pre-gelatinized starch, hydroxypropyl methyl cellulose, microcrystalline cellulose, and mixtures thereof. Suitable forms of microcrystalline cellulose include, but are not limited thereto, the materials marketed as AVICEL-PH-101, AVICEL-PH-103, AVICEL RC-581, AVICEL-PH-105 (available from FMC Corporation, American Viscose Division, Avicel Sales, Marcus Hook, Pa.), or a mixture thereof.

As used herein, “filler” suitable for use in pharmaceutical compositions include, but are not limited thereto, talc, calcium carbonate (e.g., granule or powder), microcrystalline cellulose, powdered cellulose, dextrates, kaolin, mannitol, silicic acid, sorbitol, starch, pre-gelatinized starch, or a mixture thereof.

As used herein, “diluent” suitable for use in pharmaceutical compositions include, but are not limited thereto, lactose, comprising anhydrous lactose and lactose monohydrate, starches, comprising directly compressible starch and hydrolyzed starches (e.g., Celutab™ and Emdex™), mannitol, sorbitol, xylitol, dextrose (e.g., Cerelose™ 2000) and dextrose monohydrate, dibasic calcium phosphate dihydrate, sucrose-based diluents, confectioner's sugar, monobasic calcium sulfate monohydrate, calcium sulfate dihydrate, granular calcium lactate trihydrate, dextrates, inositol, hydrolyzed cereal solids, amylose, celluloses including microcrystalline cellulose, food grade sources of α- and amorphous cellulose (e.g., Rexcel™) and powdered cellulose, calcium carbonate, glycine, bentonite, polyvinylpyrrolidone, or a mixture thereof.

The term “disintegrating agent” refers to an agent which provides tablets that disintegrate when exposed to an aqueous environment. Examples of disintegrating agents suitable for use in pharmaceutical compositions include, but are not limited thereto, starches, comprising potato starch, tapioca starch, sodium starch glycolate (e.g., Explotab™ of Pen West) and pre-gelatinized starch such as pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), calcium carbonate, polacrilin potassium, croscarmellose sodium, alginates, alginic acid, crospovidone, and gums such as agar-agar, guar, xanthan, locust bean, karaya, pectin, tragacanth gums, or a mixture thereof.

As used herein, “lubricant” suitable for use in pharmaceutical compositions include, but are not limited thereto, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Piano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

The term “glidant” refers to a substance which promotes powder flow of a solid formulation. As used herein, suitable glidants for use in pharmaceutical compositions include, but are not limited thereto, colloidal silicon dioxide, starch, talc, tribasic calcium phosphate, powdered cellulose, magnesium trisilicate, or a mixture thereof.

As used herein, “flavouring agent” suitable for use in pharmaceutical compositions comprise natural flavors extracted from a natural product such as plant, fruit, or dairy product, an edible synthetic compound which produces a pleasant taste sensation, or a mixture thereof. Examples of flavouring agents include, but are not limited thereto, acacia syrup, alitame, anise, apple, aspartame, banana, berry, black currant, butter, butter pecan, butterscotch, calcium citrate, camphor, caramel, cherry, cherry cream, chocolate, cinnamon, citrus, citrus punch, citrus cream, cocoa, coffee, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, MagnaSweet®, maltol, mannitol, maple, menthol, mint, mint cream, mixed berry, nut, orange, peanut butter, pear, peppermint, peppermint cream, Prosweet® Powder, raspberry, root beer, rum, saccharin, safrole, sorbitol, spearmint, spearmint cream, strawberry, strawberry cream, stevia, sucralose, sucrose, tagatose, tangerine, thaumatin, tutti fruitti, vanilla, walnut, watermelon, wild cherry, wintergreen, xylitol, or mixtures thereof.

As used herein, “wetting agent” suitable for use in pharmaceutical compositions include, but are not limited thereto, quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene caprylic/capric mono- and diglycerides (e.g., Labrasol™ of Gattefosse), polyoxyethylene castor oil and polyoxyethylene hydrogenated castor oil; polyoxyethylene alkyl ethers, for example polyoxyethylene cetostearyl ether, polyoxyethylene fatty acid esters, for example polyoxyethylene stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, polyoxyethylene lauryl ether, fatty acids and salts thereof, for example oleic acid, sodium oleate and triethanolamine oleate, glyceryl fatty acid esters, for example glyceryl monostearate, propylene glycol monostearate, sorbitan esters, for example sorbitan monolaurate, sorbitan monooleate, diethylene glycol monolaurate, sorbitan monopalmitate and sorbitan monostearate, tyloxapol, or mixtures thereof.

As used herein, “emulsifying agent” suitable for use in pharmaceutical compositions include, but are not limited thereto, mono- and diglycerides of fatty acids, esters of monoglycerides of fatty acids, DATEM (E472e, diacetyl tartaric acid esters of mono- and diglycerides), propylene glycol monoesters, lecithin, hydroxylated lecithin, dioctyl sodium sulphosuccinate, SSL (E481, sodium stearoyl-2-lactylate), CSL (E482, calcium stearoyl-2-lactylate), Polysorbate 20, Polysorbate 40, Polysorbate 80, Polysorbate 80, sorbitan tristearate, stearyi citrate, PGPR (polyglycerin-polyricinoleat), caseinate, gelatin, acacia, tragacanth, bentonite, surfactants such as polyoxyethylene sorbitan monooleate, or mixtures thereof.

As used herein, “pH buffering agent” (also referred to as buffering agents) suitable for use in pharmaceutical compositions include, but are not limited thereto, a salt of a Group IA metal, including, for example a bicarbonate salt of a Group IA metal, a carbonate salt of a Group IA metal, an alkali earth metal buffering agent, an aluminum buffering agent, a calcium buffering agent, or a magnesium buffering agent. Other suitable pH buffering agents include alkali (sodium and potassium) or alkali earth (calcium and magnesium) carbonates, phosphates, bicarbonates, citrates, borates, acetates, phthalates, tarfrates, succinates and the like, such as, for example, sodium or potassium phosphate, citrate, borate, acetate, bicarbonate and carbonate, an amino acid, an acid salt of an amino acid, an alkali salt of an amino acid, aluminum hydroxide, aluminum hydroxide/magnesium carbonate, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate coprecipitate, aluminum glycinate, calcium acetate, calcium bicarbonate, calcium borate, calcium carbonate, calcium citrate, calcium chloride, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium dihydrogen phosphate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, trihydroxymethylaminomethane, tripotassium phosphate, trisodium phosphate, and trometamol. (Based in part upon the list provided in The Merck Index, Merck & Co. Rahway, N.J. (2001)).

As used herein, “sweetening agent” suitable for use in suitable for use in pharmaceutical compositions comprise natural or artificial agents, which introduce or enhance a sweet taste. Examples of sweetening agents include, but are not limited thereto, acesulfame potassium (acesulfame K), alitame, aspartame, cyclamate, cylamate, dextrose, isomalt, glycerin, MagnaSweet®, maltitol, mannitol, neohesperidine DC, neotame, Prosweet® Powder, saccharin, sorbitol, stevia, sucralose, sucrose, tagatose, thaumatin, xylitol, or mixtures thereof.

The phrase “effective amount”, “effective dosage”, or “effective dosage amount”, when used in connection with another therapeutic agent or a second therapeutic agent means an amount for providing the therapeutic effect of the second therapeutic agent. The phrase “effective amount”, “effective dosage”, or “effective dosage amount”, when used in connection with Etifoxine means an amount for providing the therapeutic effect of Etifoxine, or a pharmaceutically acceptable derivative thereof.

As used herein, the terms “cancer” and “cancerous” refer to or describe the physiological condition in a subject, e.g., mammal, in which a population of cells are characterized by unregulated cell growth. “Cancer cells” and “tumor cells” as used herein refer to the total population of cells derived from a tumor including both non-tumorigenic cells, which comprise the bulk of the tumor cell population, and tumorigenic stem cells (cancer stem cells). Examples of cancer include, but are not limited to, carcinoma, lymphoma, blastoma, sarcoma, and leukemia. More particular examples of cancer is selected from the group consisting of breast cancer, head and neck cancer, lung cancer, ovarian cancer, pancreatic cancer, prostate cancer, renal cancer, skin cancer, hepatocellular carcinoma, esophageal cancer, cervical cancer, glioma, bladder cancer, endometrial cancer, bile duct cancer, bone cancer, retinoblastoma, gallbladder cancer, pituitary cancer, rectal cancer, salivary gland cancer, nasal pharyngeal, sarcoma, brain cancer, gastric cancer, multiple myeloma, leukemia, thyroid cancer, and lymphoma, Hodgkin lymphoma, mast cell sarcoma, mastocytoma, mast cell leukaemia, melanoma, Merkel cell carcinoma, neurofibromatosis, pancreatic carcinoma, squamous cell cancer, small-cell lung cancer, non-small cell lung cancer, adenocarcinoma of the lung, squamous carcinoma of the lung, cancer of the peritoneum, gastrointestinal cancer, glioblastoma, liver cancer, hepatoma, colon cancer, colorectal cancer, endometrial carcinoma, uterine carcinoma, salivary gland carcinoma, kidney cancer, vulvar cancer, hepatic carcinoma, and various types of head and neck cancer.

The expression “anticancer drug” or “anticancer agent” refers to any chemotherapeutic, immunotherapeutic or immunomodulatory, antiangiogenic, hormonal or naturally occurring, semi-synthetic or synthetic therapeutic drug or agent effective to treat or prevent cancer. Representative examples of anticancer agents are listed below.

“Tumor” as used herein refers to any mass of tissue that result from excessive cell growth or proliferation, either benign (noncancerous) or malignant (cancerous) including precancerous lesions.

“Metastasis” or “metastatic cancer” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

As used herein, the term “autoimmune disease” or “autoimmune disorder” means a disease or disorder arising from an abnormal immune response directed against an individual's own tissues (i.e. autoimmunity). In a series of diseases, such as rheumatoid arthritis, inflammatory bowel disease, or diabetes, autoimmune disorder is closely associated with an inflammation disorder. Autoimmune disorders may or may not be associated with inflammation. Therefore, certain disorders may be characterized as both autoimmune and inflammatory disorders. Examples of autoimmune disorders include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, thyroid-associated ophthalmopathy (TAO) such as Grave's disease, Wegener's granulomatosis, and the like.

As used herein, the term “inflammatory disease” or “inflammatory disorder” refers to any disease, condition or disorder associated with inflammation, preferably chronic inflammation. In a series of diseases, such as rheumatoid arthritis, inflammatory bowel disease, or diabetes, inflammation disorder is closely associated with an autoimmune disorder. Inflammation may or may not be caused by an autoimmune disorder. Thus, certain disorders may be characterized as both autoimmune and inflammatory disorders. An inflammatory disorder may arise from pathogens, external injuries (e.g., scrapes), chemical exposition, radiation, or may result from an autoimmune disease including, but not limited to, cystitis, or dermatitis. Examples of inflammatory disorders include, but are not limited to, asthma, encephilitis, inflammatory bowel disease, chronic obstructive pulmonary disease (COPD), allergic disorders, septic shock, pulmonary fibrosis, undifferentiated spondyloarthropathy, undifferentiated arthropathy, arthritis, cachexia, inflammatory osteolysis, renal inflammation, eosinophilic colitis, atopic dermatitis, eosinophilic gastritis, eosinophilic esophagitis, and chronic inflammation resulting from chronic viral, bacteria infections, and the like, preferably encephilitis, inflammatory bowel disease, chronic obstructive pulmonary disease (COPD), allergic disorders, septic shock, pulmonary fibrosis, undifferentiated spondyloarthropathy, undifferentiated arthropathy, arthritis, cachexia, inflammatory osteolysis, renal inflammation, eosinophilic colitis, atopic dermatitis, eosinophilic gastritis, eosinophilic esophagitis, and chronic inflammation resulting from chronic viral, bacteria infections, and the like.

As used herein, the term “fibrotic disorder” or “fibrotic disease” refers to any disease, condition or disorder associated with fibrosis (i.e. scarring). Fibrosis means the accumulation of excess fibrous connective tissue in an organ or tissue undergoing a reparative or reactive process. Examples of a fibrotic disorder or fibrotic disease include, but not limited thereto, acute and chronic forms of fibrosis of organs, comprising etiological variations of pulmonary fibrosis (comprising interstitial lung disease and fibrotic lung disease), liver fibrosis, cardiac fibrosis (comprising myocardial fibrosis), kidney fibrosis including chronic renal failure, skin fibrosis (comprising Scleroderma), keloids and hypertrophic Scars, bone marrow fibrosis (comprising myelofibrosis). Further Examples of fibrotic disorders encompass various types of ocular scarring (comprising proliferative vitreoretinopathy and scarring resulting from Surgery to treat cataract or glaucoma), inflammatory bowel disease of variable etiology, macular degeneration, Grave's ophthalmopathy, drug induced ergotism, psoriasis, cystic fibrosis, glioblastoma in Li-Fraumeni Syndrome, Sporadic glioblastoma, myleoid leukemia, acute myelogenious leukemia, myelodysplastic Syndrome, myeloproferative Syndrome, gynecological cancer, Kaposi's Sarcoma, Hansen's disease, collagenous colitis, and the like, preferably acute and chronic forms of fibrosis of organs, comprising etiological variations of pulmonary fibrosis (comprising interstitial lung disease and fibrotic lung disease), liver fibrosis, cardiac fibrosis (comprising myocardial fibrosis), kidney fibrosis including chronic renal failure, skin fibrosis (comprising Scleroderma), keloids and hypertrophic Scars, bone marrow fibrosis (comprising myelofibrosis). Further Examples of fibrotic disorders encompass various types of ocular scarring (comprising proliferative vitreoretinopathy and scarring resulting from Surgery to treat cataract or glaucoma), inflammatory bowel disease of variable etiology, macular degeneration, Grave's ophthalmopathy, drug induced ergotism, psoriasis, cystic fibrosis, Hansen's disease, collagenous colitis, and the like.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Terms such as “treating” or “treatment” or “to treat” as used herein refer to both 1) therapeutic measures that cure, slow down, lessen symptoms of, and/or halt progression of a diagnosed pathologic condition or disorder, and 2) prophylactic or preventative measures that prevent or slow the development of a targeted pathologic condition or disorder. Thus those in need of treatment include those already with the disorder; those prone to have the disorder; and those in whom the disorder is to be prevented. A subject is successfully “treated” according to the methods of the present disclosure if the patient shows one or more of the following: a reduction in the number of or complete absence of cancer cells; a reduction in the tumor size; inhibition of or an absence of cancer cell infiltration into peripheral organs including the spread of cancer into soft tissue and bone; inhibition of or an absence of tumor metastasis; inhibition or an absence of tumor growth; relief of one or more symptoms associated with the specific cancer; reduced symptoms, reduced morbidity and mortality; and improvement in quality of life, preferably reduced symptoms, reduced morbidity and mortality; and improvement in quality of life.

As used herein, the term “inhibiting” or “to inhibit” and their grammatical equivalents, when used in the context of a bioactivity, refer to a down-regulation of the bioactivity, which may reduce or eliminate the targeted function, such as the production of a protein or the phosphorylation of a molecule. When used in the context of mast cells, the terms refer to a decrease of degranulation and release of mediators from the mast cells. When used in the context of a disorder or disease, the terms refer to success at preventing the onset of symptoms, alleviating symptoms, or eliminating the disease, condition or disorder.

In general, the term “parenteral administration” encompasses intravenous administration, intramuscular administration, subcutaneous administration and intradermal administration.

The term “intravenous administration” refers to a type of administration of a therapeutic agent, wherein the therapeutic agent is typically in liquid form and is administered directly into a vein. Examples of subcutaneous administration of a therapeutic agent include, but are not limited thereto, injection, infusion, and the like.

The term “intramuscular administration” refers to a type of administration of a therapeutic agent, wherein the therapeutic agent is administered directly into the muscle. Examples of intramuscular administration of a therapeutic agent include, but are not limited thereto, injection, and the like.

The term “subcutaneous administration” refers to a type of administration of a therapeutic agent, wherein the therapeutic agent is administered into the subcutis, the layer of skin below the epidermis and dermis. Examples of subcutaneous administration of a therapeutic agent include, but are not limited thereto, injection, and the like.

The term “intradermal administration” (also referred to as “intracutaneous administration”) refers to a type of administration of a therapeutic agent, wherein the therapeutic agent is administered into the dermis, which is located just below the epidermis. Examples of intradermal administration of a therapeutic agent include, but are not limited thereto, injection, and the like.

The terms “mucosal administration” (also referred to as “transmucosal administration”) refer to a type of administration of a therapeutic agent, wherein the therapeutic agent is administered into the mucosa of different kinds of body parts such as nose, mouth, rectum or vagina. Examples of mucosal administration of a therapeutic agent include, but are not limited thereto, sublingual tablet, suppository, aerosol, nasal solution, and the like.

The term “oral administration” refers to a type of administration of a therapeutic agent, wherein the therapeutic agent is administered orally (i.e., by mouth). Specific types of oral administration include, but not limited thereto, “sublingual administration”, and “buccal administration”. Examples of oral administration of a therapeutic agent include, but are not limited thereto, tablet, dispersible powder, granule, capsule, syrup, elixir, suspension, and the like.

The term “sublingual administration” as used herein belongs to the route of “mucosal administration”, more specifically to the route of “oral administration”, and refers to a type of administration of a therapeutic agent, wherein the therapeutic agent is administered under the tongue. Examples of sublingual administration of a therapeutic agent include, but are not limited thereto, tablet, capsule, and the like.

The term “buccal administration” as used herein belongs to the route of “mucosal administration”, more specifically to the route of “oral administration”, and refers to a type of administration of a therapeutic agent, wherein the therapeutic agent is administered between the gingiva and the cheek. Examples of buccal administration of a therapeutic agent include, but are not limited thereto, tablet, capsule, and the like.

The term “intraperitoneal administration” refers to a type of administration of a therapeutic agent, wherein the therapeutic agent is administered directly into the peritoneum. Examples of intraperitoneal administration of a therapeutic agent include, but are not limited thereto, injection, and the like.

The term “intranasal administration” (also referred to as “nasal administration”) as used herein refers to a type of administration of a therapeutic agent, wherein the therapeutic agent is administered into the nose. Examples of intranasal administration of a therapeutic agent include, but are not limited thereto, nasal drops, nasal sprays, and the like.

The term “rectal administration” refers to a type of administration of a therapeutic agent, wherein the therapeutic agent is administered into the rectum. Examples of rectal administration of a therapeutic agent include, but are not limited thereto, tablet, capsule, rectal catheter, and the like.

The term “transdermal administration” refers to a type of administration of a therapeutic agent, wherein the therapeutic agent is administered on the skin resulting in the penetration of the therapeutic agent into the skin layers (i.e., absorption). Examples of intramuscular administration of a therapeutic agent include, but are not limited thereto, transdermal patches, and the like.

The term “topical administration” refers to a type of administration of a therapeutic agent, wherein the therapeutic agent is applied topically (i.e., locally) on the skin and the pharmacodynamic effect thereof is local. Examples of topical administration of a therapeutic agent include, but are not limited thereto, creme, ointment, and the like.

The term “intravaginal administration” refers to a type of administration of a therapeutic agent, wherein the therapeutic agent is administered into the vagina. Examples of intravaginal administration of a therapeutic agent include, but are not limited thereto, tablet, capsule, and the like.

The term “gastrointestinal administration” (also referred to as “enteral administration” or “enteric administration”) refers to a type of administration of a therapeutic agent, wherein the therapeutic agent is administered in a way which results in an uptake of the therapeutic agent by the gastrointestinal tract. Gastrointestinal administration therefore comprises oral administration, rectal administration, and the like.

The term “administration by inhalation” refers to a type of administration of a therapeutic agent, wherein the therapeutic agent is administered by inhalation. Examples of administration of a therapeutic agent by inhaling include, but are not limited thereto, inhaler device, aerosol, and the like.

As used herein, the term “in vitro” refers to a method performed in intact cells in an artificial environment created outside a living multicellular organism (e.g., a test tube or culture plate).

As used herein, the term “ex vivo” refers to a method performed on living cells or tissue in an artificial environment outside the organism, wherein the living cells or tissue were removed from a living organism.

As used herein, the term “degranulation” or “mast cell degranulation” refers to biological processes of mast cells including, but not limited thereto, “secretion”, “excretion”, “exocytosis”, “degranulation” according to the concrete definition, or a mixture thereof. “Secretion” involves the release of substances from one place of containment to another, i.e., from a cell to its extracellular environment or a gland to the skin's surface. “Excretion” means the elimination of a waste material from a cell or an organ. “Exocytosis” means a process of cellular secretion or excretion in which substances contained in vesicles are discharged from the cell by fusion of the vesicular membrane with the outer cell membrane. According to the concrete definition, degranulation refers to the loss of or release of granules from mast cells. Unless specified otherwise (such as “degranulation according to the concrete definition”), the term degranulation or mast cell degranulation, as used herein, is to be understood in the general sense, i.e., degranulation or mast cell degranulation comprising secretion, excretion, exocytosis, degranulation according to the concrete definition, or a mixture thereof.

The term “mediators” (also referred to as “mast cell mediators”) as used herein, includes, but is not limited thereto, de novo mediators (also referred to as “newly synthesized mediators”), i.e., newly synthesized substances upon mast cell activation, and/or preformed mediators (also referred to as “pre-stored mediators”), i.e., preformed substances stored in granules and released from the mast cell upon mast cell activation. Typical preformed mediators comprise histamine, heparin, proteases, tryptases, chymases, cathepsin G-like enzymes, elastases, carboxypeptidase A, and acidic hydrolases. Typical de novo mediators comprise arachidonic acid, prostaglandin D2, leukotrienes C4 and B4, and interleukins (e.g., IL-3).

As used herein, in the context of mast cells, the terms “activation” and “stimulation” are used interchangeably. The term “activated mast cells” (i.e., “stimulated mast cells”) or “mast cell activation” (i.e., “mast cell stimulation”) refer to activation of mast cells by at least one kind of a variety of endogenous and/or exogenous agents, leading to degranulation of the stimulated mast cells. The term degranulation of an activated mast cell is used equally to the term of activation of a mast cell with resulting release of preformed and stored mediators and de nove mediators. Mast cells may become immunologically activated by binding of antigen to IgE on a mast cell surface. In addition to the classical IgE-dependent mast cell activation, mast cell activation can be induced via IgE-independent pathways. These include i.a. mast cell activation by stem cell factor (SCF), Nerve Growth Factor (NGF) or polybasic compounds [Piliponsky A. M. et al. (2003) Blood 101:1898-4; Feldweg, A. M. et al. (2003) Eur. J. Immunol. 33:2262-8].

### Etifoxine for Use in the Treatment of a Disease Related to Mast Cell Activation

The present disclosure relates to Etifoxine, or a pharmaceutically acceptable derivative thereof, for use in the treatment of a disease related to activated mast cells in a subject. In a preferred embodiment, the present disclosure relates to Etifoxine, or a pharmaceutically acceptable derivative thereof, for use in the treatment of a disease related to activated mast cells in a human.

As used herein, the term “Etifoxine” refers to “Etifoxine, or a pharmaceutically acceptable derivative thereof”.

In a preferred embodiment, the present disclosure relates to Etifoxine for use in the treatment of a disease related to activated mast cells, wherein the disease is selected from the group consisting of asthma, allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, H1N1 influenza, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, tumor growth, metastasis, cancer, autoimmune disorders, inflammatory disorders, and fibrotic disorders, preferably wherein the disease is selected from the group consisting of allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, H1N1 influenza, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, autoimmune disorders, inflammatory disorders, and fibrotic disorders.

In a preferred embodiment, the present disclosure relates to Etifoxine for use in the treatment of a disease related to activated mast cells, wherein the disease is selected from the group consisting of viral hemorrhagic fever caused by a virus, wherein the virus is a member of at least one of the virus families Arenaviridae, Filoviridae, Bunyaviridae, Flaviviridae, and Rhabdoviridae. In a preferred embodiment, the virus comprises Dengue virus, Ebola virus, Marburg virus, Hanta virus, West Nile virus, Saint Louis encephalitis, Kunjin virus, Lassa virus, Crimean-Congo hemorrhagic fever virus, Rift Valley fever virus, yellow fever virus, Japanese encephalitis virus, Lujo virus, Junin virus, Argentine hemorrhagic fever virus, Brazilian hemorrhagic fever virus, Bolivian hemorrhagic fever virus, Argentine hemorrhagic fever virus, Garissa virus, SFTS virus, Hantan virus, Omsk hemorrhagic fever virus, Kyasanur forest disease virus, Langat virus, Alkhurma virus, or Henipa virus.

In a preferred embodiment, the present disclosure relates to Etifoxine for use in the treatment of a disease related to activated mast cells, wherein the disease is selected from the group consisting of viral infections, wherein the virus is a member of at least one virus family selected from the group consisting of Coronaviridae and Orthomyxoviridae. In a preferred embodiment, the virus comprises MERS-CoV, SARS-CoV, particularly SARS-CoV-2, influenza A virus, or H1N1 influenza. In this context, SARS-CoV refers to the virus strain (virus family), comprising SARS-CoV-1 (sometimes also refered to as SARS-CoV) and SARS-CoV-2.

In another preferred embodiment, the present disclosure relates to Etifoxine for use in the treatment of a disease related to activated mast cells, wherein the disease is selected from the group consisting of allergy, asthma, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, tumor growth, metastasis, cancer, fibrotic disorders, and autoimmune disorders, preferably wherein the disease is selected from the group consisting of allergy, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, fibrotic disorders, and autoimmune disorders.

In another preferred embodiment, the present disclosure relates to Etifoxine for use in the treatment of a disease related to activated mast cells, wherein the disease is selected from the group consisting of viral hemorrhagic fever caused by a virus comprising Dengue virus, West Nile virus, Crimean-Congo hemorrhagic fever virus, or Japanese encephalitis virus.

In another preferred embodiment, the present disclosure relates to Etifoxine for use in the treatment of a disease related to activated mast cells, wherein the disease is selected from the group consisting of viral infection caused by a virus comprising MERS-CoV, SARS-CoV, SARS-CoV-2, influenza A, or H1N1 influenza. In this context, SARS-CoV refers to the virus strain (virus family), comprising SARS-CoV-1 (sometimes also refered to as SARS-CoV) and SARS-CoV-2.

In another preferred embodiment, the present disclosure relates to Etifoxine for use in the treatment of a disease related to activated mast cells, wherein the disease is selected from the group consisting of allergy, asthma, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, systemic mast cell activation disease, fibrotic disorders, tumor growth, metastasis, and cancer, preferably wherein the disease is selected from the group consisting of allergy, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, systemic mast cell activation disease and fibrotic disorders. In a more preferred embodiment, the present disclosure relates to Etifoxine for use in the treatment of a disease related to activated mast cells, wherein the disease is selected from the group consisting of allergy, asthma, autoimmune disorders, chronic fatigue syndrome, chronic lyme disease, fibromyalgia, fibrotic disorders, inflammatory bowel disease, irritable bowel syndrome, systemic mastocytosis, and systemic mast cell activation disease, preferably wherein the disease is selected from the group consisting of allergy, autoimmune disorders, chronic fatigue syndrome, chronic lyme disease, fibromyalgia, fibrotic disorders, inflammatory bowel disease, irritable bowel syndrome, systemic mastocytosis, and systemic mast cell activation disease.

In a most preferred embodiment, the present disclosure relates to Etifoxine for use in the treatment of a disease related to activated mast cells, wherein the disease is selected from the group consisting of allergy, asthma, fibromyalgia, fibrotic disorders, inflammatory bowel disease, irritable bowel syndrome, systemic mastocytosis, and systemic mast cell activation disease, preferably wherein the disease is selected from the group consisting of allergy, fibromyalgia, fibrotic disorders, inflammatory bowel disease, irritable bowel syndrome, systemic mastocytosis, and systemic mast cell activation disease.

In another most preferred embodiment, the present invention relates to Etifoxine for use in the treatment of a disease related to activated mast cells, wherein the disease is selected from the group consisting of allergy, atherosclerosis, bladder pain syndrome, chronic fatigue syndrome, chronic lyme disease, colitis ulcerosa, Dengue fever, fibromyalgia, fibrotic disease, interstitial cystitis, irritable bowel syndrome, obesity, SARS-CoV-2 (Covid-19), systemic mast cell activation syndrome, systemic lupus erythematosus, thrombotic disease, urticarial, viral infections and vulvodynia.

In another most preferred embodiment, the present disclosure relates to Etifoxine for use in the treatment of a disease related to activated mast cells, wherein the disease is selected from the group consisting of viral hemorrhagic fever caused by Dengue virus. In a preferred embodiment, Etifoxine is present as enatiomerically pure R-Etifoxine. In another preferred embodiment, Etifoxine is present as enatiomerically pure S-Etifoxine. In another preferred embodiment, Etifoxine is racemic (i.e., present in a mixture of equal parts of R-Etifoxine and S-Etifoxine).

In another preferred embodiment, the present disclosure relates to Etifoxine in the form of S-Etifoxine for use in the treatment of a disease related to activated mast cells, wherein the disease is selected from the group consisting of asthma, allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, H1N1 influenza, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, tumor growth, metastasis, cancer, autoimmune disorders, inflammatory disorders, and fibrotic disorders, preferably wherein the disease is selected from the group consisting of allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, H1N1 influenza, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, autoimmune disorders, inflammatory disorders, and fibrotic disorders.

In another preferred embodiment, the present disclosure relates to Etifoxine in the form of S-Etifoxine for use in the treatment of a disease related to activated mast cells, wherein the disease is selected from the group consisting of viral hemorrhagic fever caused by a virus, wherein the virus is a member of at least one of the virus families Arenaviridae, Filoviridae, Bunyaviridae, Flaviviridae, and Rhabdoviridae. In a preferred embodiment, the virus comprises Dengue virus, Ebola virus, Marburg virus, Hanta virus, West Nile virus, Saint Louis encephalitis, Kunjin virus, Lassa virus, Crimean-Congo hemorrhagic fever virus, Rift Valley fever virus, yellow fever virus, Japanese encephalitis virus, Lujo virus, Junin virus, Argentine hemorrhagic fever virus, Brazilian hemorrhagic fever virus, Bolivian hemorrhagic fever virus, Argentine hemorrhagic fever virus, Garissa virus, SFTS virus, Hantan virus, Omsk hemorrhagic fever virus, Kyasanur forest disease virus, Langat virus, Alkhurma virus, or Henipa virus.

In another preferred embodiment, the present disclosure relates to Etifoxine in the form of S-Etifoxin for use in the treatment of a disease related to activated mast cells, wherein the disease is selected from the group consisting of viral infections, wherein the virus is a member of at least one virus family selected from the group consisting of Coronaviridae and Orthomyxoviridae. In a preferred embodiment, the virus comprises MERS-CoV, SARS-CoV, SARS-CoV-2, influenza A, or H1N1 influenza.

In another preferred embodiment, the present disclosure relates to Etifoxine in the form of S-Etifoxine for use in the treatment of a disease related to activated mast cells, wherein the disease is selected from the group consisting of allergy, asthma, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, tumor growth, metastasis, cancer, fibrotic disorders, and autoimmune disorders, preferably wherein the disease is selected from the group consisting of allergy, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, fibrotic disorders, and autoimmune disorders.

In another preferred embodiment, the present disclosure relates to Etifoxine in the form of S-Etifoxine for use in the treatment of a disease related to activated mast cells, wherein the disease is selected from the group consisting of viral hemorrhagic fever caused by a virus comprising Dengue virus, West Nile virus, Crimean-Congo hemorrhagic fever virus, or Japanese encephalitis virus.

In another preferred embodiment, the present disclosure relates to Etifoxine in the form of S-Etifoxin for use in the treatment of a disease related to activated mast cells, wherein the disease is selected from the group consisting of viral infection caused by a virus comprising MERS-CoV, SARS-CoV, SARS-CoV-2, influenza A, or H1N1 influenza.

In another preferred embodiment, the present disclosure relates to Etifoxine in the form of S-Etifoxine for use in the treatment of a disease related to activated mast cells, wherein the disease is selected from the group consisting of allergy, asthma, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, systemic mast cell activation disease, fibrotic disorders, tumor growth, metastasis, and cancer, preferably wherein the disease is selected from the group consisting of allergy, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, systemic mast cell activation disease, and fibrotic disorders.

In a more preferred embodiment, the present disclosure relates to Etifoxine in the form of S-Etifoxine for use in the treatment of a disease related to activated mast cells, wherein the disease is selected from the group consisting of allergy, asthma, autoimmune disorders, chronic fatigue syndrome, chronic lyme disease, fibromyalgia, fibrotic disorders, inflammatory bowel disease, irritable bowel syndrome, systemic mastocytosis, and systemic mast cell activation disease, preferably wherein the disease is selected from the group consisting of allergy, autoimmune disorders, chronic fatigue syndrome, chronic lyme disease, fibromyalgia, fibrotic disorders, inflammatory bowel disease, irritable bowel syndrome, systemic mastocytosis, and systemic mast cell activation disease.

In a most preferred embodiment, the present disclosure relates to Etifoxine in the form of S-Etifoxine for use in the treatment of a disease related to activated mast cells, wherein the disease is selected from the group consisting of allergy, asthma, fibromyalgia, fibrotic disorders, inflammatory bowel disease, irritable bowel syndrome, systemic mastocytosis, and systemic mast cell activation disease, preferably wherein the disease is selected from the group consisting of allergy, fibromyalgia, fibrotic disorders, inflammatory bowel disease, irritable bowel syndrome, systemic mastocytosis, and systemic mast cell activation disease.

In another most preferred embodiment, the present disclosure relates to Etifoxine in the form of S-Etifoxine for use in the treatment of a disease related to activated mast cells, wherein the disease is selected from the group consisting of viral hemorrhagic fever caused by Dengue virus.

In another most preferred embodiment, the present invention relates to Etifoxine in the form of S-Etifoxine for use in the treatment of a disease related to activated mast cells, wherein the disease is selected from the group consisting of allergy, atherosclerosis, bladder pain syndrome, chronic fatigue syndrome, chronic lyme disease, colitis ulcerosa, Dengue fever, fibromyalgia, fibrotic disease, interstitial cystitis, irritable bowel syndrome, obesity, SARS-CoV-2 (Covid-19), systemic mast cell activation syndrome, systemic lupus erythematosus, thrombotic disease, urticarial, viral infections and vulvodynia.

In another most preferred embodiment, the present disclosure relates to Etifoxine in the form of S-Etifoxine for use in the treatment of a disease related to activated mast cells, wherein the disease is selected from the group consisting of viral infections caused by SARS-CoV-2.

In another embodiment, the present disclosure relates to Etifoxine in the form of R-Etifoxine for use in the treatment of a disease related to activated mast cells, wherein the disease is selected from the group consisting of asthma, allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, H1N1 influenza, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, tumor growth, metastasis, cancer, autoimmune disorders, inflammatory disorders, and fibrotic disorders.

In another embodiment, the present disclosure relates to Etifoxine in the form of R-Etifoxine for use in the treatment of a disease related to activated mast cells, wherein the disease is selected from the group consisting of viral hemorrhagic fever caused by a virus, wherein the virus is a member of at least one of the virus families Arenaviridae, Filoviridae, Bunyaviridae, Flaviviridae, and Rhabdoviridae. In a preferred embodiment, the virus comprises Dengue virus, Ebola virus, Marburg virus, Hanta virus, West Nile virus, Saint Louis encephalitis, Kunjin virus, Lassa virus, Crimean-Congo hemorrhagic fever virus, Rift Valley fever virus, yellow fever virus, Japanese encephalitis virus, Lujo virus, Junin virus, Argentine hemorrhagic fever virus, Brazilian hemorrhagic fever virus, Bolivian hemorrhagic fever virus, Argentine hemorrhagic fever virus, Garissa virus, SFTS virus, Hantan virus, Omsk hemorrhagic fever virus, Kyasanur forest disease virus, Langat virus, Alkhurma virus, or Henipa virus.

In another preferred embodiment, the present disclosure relates to Etifoxine in the form of R-Etifoxin for use in the treatment of a disease related to activated mast cells, wherein the disease is selected from the group consisting of viral infections, wherein the virus is a member of at least one virus family selected from the group consisting of Coronaviridae and Orthomyxoviridae. In a preferred embodiment, the virus comprises MERS-CoV, SARS-CoV, SARS-CoV-2, influenza A, or H1N1 influenza.

In a preferred embodiment, the present disclosure relates to Etifoxine in the form of R-Etifoxine for use in the treatment of a disease related to activated mast cells, wherein the disease is selected from the group consisting of allergy, asthma, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, tumor growth, metastasis, cancer, fibrotic disorders, and autoimmune disorders, preferably wherein the disease is selected from the group consisting of allergy, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, fibrotic disorders, and autoimmune disorders

In another preferred embodiment, the present disclosure relates to Etifoxine in the form of R-Etifoxine for use in the treatment of a disease related to activated mast cells, wherein the disease is selected from the group consisting of viral hemorrhagic fever caused by a virus comprising Dengue virus, West Nile virus, Crimean-Congo hemorrhagic fever virus, or Japanese encephalitis virus.

In another preferred embodiment, the present disclosure relates to Etifoxine in the form of R-Etifoxin for use in the treatment of a disease related to activated mast cells, wherein the disease is selected from the group consisting of viral infection caused by a virus comprising SARS-CoV-2 or H1N1 influenza.

In yet another preferred embodiment, the present disclosure relates to Etifoxine in the form of R-Etifoxine for use in the treatment of a disease related to activated mast cells, wherein the disease is selected from the group consisting of allergy, asthma, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, systemic mast cell activation disease, fibrotic disorders, tumor growth, metastasis, and cancer, preferably wherein the disease is selected from the group consisting of allergy, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, systemic mast cell activation disease and fibrotic disorders.

In a more preferred embodiment, the present disclosure relates to Etifoxine in the form of R-Etifoxine for use in the treatment of a disease related to activated mast cells, wherein the disease is selected from the group consisting of allergy, asthma, autoimmune disorders, chronic fatigue syndrome, chronic lyme disease, fibromyalgia, fibrotic disorders, inflammatory bowel disease, irritable bowel syndrome, systemic mastocytosis, and systemic mast cell activation disease, preferably wherein the disease is selected from the group consisting of allergy, autoimmune disorders, chronic fatigue syndrome, chronic lyme disease, fibromyalgia, fibrotic disorders, inflammatory bowel disease, irritable bowel syndrome, systemic mastocytosis, and systemic mast cell activation disease.

In a most preferred embodiment, the present disclosure relates to Etifoxine in the form of R-Etifoxine for use in the treatment of a disease related to activated mast cells, wherein the disease is selected from the group consisting of allergy, asthma, fibromyalgia, fibrotic disorders, inflammatory bowel disease, irritable bowel syndrome, systemic mastocytosis, and systemic mast cell activation disease, preferably wherein the disease is selected from the group consisting of allergy, fibromyalgia, fibrotic disorders, inflammatory bowel disease, irritable bowel syndrome, systemic mastocytosis, and systemic mast cell activation disease.

In another most preferred embodiment, the present invention relates to Etifoxine in the form of R-Etifoxine for use in the treatment of a disease related to activated mast cells, wherein the disease is selected from the group consisting of allergy, atherosclerosis, bladder pain syndrome, chronic fatigue syndrome, chronic lyme disease, colitis ulcerosa, Dengue fever, fibromyalgia, fibrotic disease, interstitial cystitis, irritable bowel syndrome, obesity, SARS-CoV-2 (Covid-19), systemic mast cell activation syndrome, systemic lupus erythematosus, thrombotic disease, urticarial, viral infections and vulvodynia. In another most preferred embodiment, the present disclosure relates to Etifoxine in the form of R-Etifoxine for use in the treatment of a disease related to activated mast cells, wherein the disease is selected from the group consisting of viral hemorrhagic fever caused by Dengue virus.

In another most preferred embodiment, the present disclosure relates to Etifoxine in the form of R-Etifoxine for use in the treatment of a disease related to activated mast cells, wherein the disease is selected from the group consisting of viral infections caused by SARS-CoV-2.

In a preferred embodiment, the present disclosure relates to Etifoxine in a racemic form for use in the treatment of a disease related to activated mast cells, wherein the disease is selected from the group consisting of asthma, allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, H1N1 influenza, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, tumor growth, metastasis, cancer, autoimmune disorders, inflammatory disorders, and fibrotic disorders, preferably wherein the disease is selected from the group consisting of allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, autoimmune disorders, inflammatory disorders, and fibrotic disorders.

In another preferred embodiment, the present disclosure relates to Etifoxine in a racemic form for use in the treatment of a disease related to activated mast cells, wherein the disease is selected from the group consisting of viral hemorrhagic fever caused by a virus, wherein the virus is a member of at least one of the virus families Arenaviridae, Filoviridae, Bunyaviridae, Flaviviridae, and Rhabdoviridae. In a preferred embodiment, the virus comprises Dengue virus, Ebola virus, Marburg virus, Hanta virus, West Nile virus, Saint Louis encephalitis, Kunjin virus, Lassa virus, Crimean-Congo hemorrhagic fever virus, Rift Valley fever virus, yellow fever virus, Japanese encephalitis virus, Lujo virus, Junin virus, Argentine hemorrhagic fever virus, Brazilian hemorrhagic fever virus, Bolivian hemorrhagic fever virus, Argentine hemorrhagic fever virus, Garissa virus, SFTS virus, Hantan virus, Omsk hemorrhagic fever virus, Kyasanur forest disease virus, Langat virus, Alkhurma virus, or Henipa virus.

In another preferred embodiment, the present disclosure relates to Etifoxine in a racemic form for use in the treatment of a disease related to activated mast cells, wherein the disease is selected from the group consisting of allergy, asthma, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, tumor growth, metastasis, cancer, fibrotic disorders, and autoimmune disorders, preferably wherein the disease is selected from the group consisting of allergy, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, fibrotic disorders, and autoimmune disorders.

In another preferred embodiment, the present disclosure relates to Etifoxine in a racemic form for use in the treatment of a disease related to activated mast cells, wherein the disease is selected from the group consisting of viral hemorrhagic fever caused by a virus comprising Dengue virus, West Nile virus, Crimean-Congo hemorrhagic fever virus, or Japanese encephalitis virus.

In another preferred embodiment, the present disclosure relates to Etifoxine in a racemic form for use in the treatment of a disease related to activated mast cells, wherein the disease is selected from the group consisting of viral infections, wherein the virus is a member of at least one virus family selected from the group consisting of Coronaviridae and Orthomyxoviridae. In a preferred embodiment, the virus comprises MERS-CoV, SARS-CoV, SARS-CoV-2, influenza A, or H1N1 influenza.

In another preferred embodiment, the present disclosure relates to Etifoxine in a racemic form for use in the treatment of a disease related to activated mast cells, wherein the disease is selected from the group consisting of allergy, asthma, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, systemic mast cell activation disease, fibrotic disorders, tumor growth, metastasis, and cancer, preferably wherein the disease is selected from the group consisting of allergy, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, systemic mast cell activation disease and fibrotic disorders.

In another preferred embodiment, the present disclosure relates to Etifoxine in a racemic form for use in the treatment of a disease related to activated mast cells, wherein the disease is selected from the group consisting of viral hemorrhagic fever caused by a virus comprising Dengue virus, West Nile virus, Crimean-Congo hemorrhagic fever virus, or Japanese encephalitis virus.

In another preferred embodiment, the present disclosure relates to Etifoxine in a racemic form for use in the treatment of a disease related to activated mast cells, wherein the disease is selected from the group consisting of viral infection caused by a virus comprising SARS-CoV-2 or H1N1 influenza.

In a more preferred embodiment, the present disclosure relates to Etifoxine in a racemic form for use in the treatment of a disease related to activated mast cells, wherein the disease is selected from the group consisting of allergy, asthma, autoimmune disorders, chronic fatigue syndrome, chronic lyme disease, fibromyalgia, fibrotic disorders, inflammatory bowel disease, irritable bowel syndrome, systemic mastocytosis, and systemic mast cell activation disease, preferably wherein the disease is selected from the group consisting of allergy, autoimmune disorders, chronic fatigue syndrome, chronic lyme disease, fibromyalgia, fibrotic disorders, inflammatory bowel disease, irritable bowel syndrome, systemic mastocytosis, and systemic mast cell activation disease.

In a most preferred embodiment, the present disclosure relates to Etifoxine in a racemic form for use in the treatment of a disease related to activated mast cells, wherein the disease is selected from the group consisting of allergy, asthma, fibromyalgia, fibrotic disorders, inflammatory bowel disease, irritable bowel syndrome, systemic mastocytosis, and systemic mast cell activation disease, preferably wherein the disease is selected from the group consisting of allergy, fibromyalgia, fibrotic disorders, inflammatory bowel disease, irritable bowel syndrome, systemic mastocytosis, and systemic mast cell activation disease.

In another most preferred embodiment, the present invention relates to Etifoxine in a racemic form use in the treatment of a disease related to activated mast cells, wherein the disease is selected from the group consisting of allergy, atherosclerosis, bladder pain syndrome, chronic fatigue syndrome, chronic lyme disease, colitis ulcerosa, Dengue fever, fibromyalgia, fibrotic disease, interstitial cystitis, irritable bowel syndrome, obesity, SARS-CoV-2 (Covid-19), systemic mast cell activation syndrome, systemic lupus erythematosus, thrombotic disease, urticarial, viral infections and vulvodynia.

In another most preferred embodiment, the present disclosure relates to Etifoxine in a racemic form for use in the treatment of a disease related to activated mast cells, wherein the disease is selected from the group consisting of viral hemorrhagic fever caused by Dengue virus.

In another most preferred embodiment, the present disclosure relates to Etifoxine in a racemic form for use in the treatment of a disease related to activated mast cells, wherein the disease is selected from the group consisting of viral infections caused by SARS-CoV-2.

In another preferred embodiment, the present disclosure relates to Etifoxine for use in the treatment of a disease related to activated mast cells, wherein the treatment comprises oral, parenteral, subcutaneous, intravenous, mucosal, intracutaneous, intramuscular, intraperitoneal, transdermal, intranasal, transmucosal, rectal, sublingual, topical, intravaginal, gastrointestinal, buccal administration, or administration by inhalation, preferably wherein the treatment comprises oral, sublingual, buccal, parenteral, intravenous, subcutaneous, topical, intravaginal, transdermal administration, or administration by inhalation, more preferably wherein the treatment comprises oral, parenteral, topical, transdermal administration, or administration by inhalation.

In another preferred embodiment, the present disclosure relates to Etifoxine in the form of R-Etifoxine for use in the treatment of a disease related to activated mast cells, wherein the treatment comprises oral, parenteral, subcutaneous, intravenous, mucosal, intracutaneous, intramuscular, intraperitoneal, transdermal, intranasal, transmucosal, rectal, sublingual, intravaginal, gastrointestinal, topical, buccal administration, or administration by inhalation. In a more preferred embodiment, the present disclosure relates to Etifoxine in the form of R-Etifoxine for use in the treatment of a disease related to activated mast cells, wherein the treatment comprises oral, sublingual, buccal, parenteral, intravenous, subcutaneous, topical, intravaginal, transdermal administration, or administration by inhalation. In a most preferred embodiment, the present disclosure relates to Etifoxine in the form of R-Etifoxine for use in the treatment of a disease related to activated mast cells, wherein the treatment comprises oral, parenteral, topical, transdermal administration, or administration by inhalation.

In another preferred embodiment, the present disclosure relates to Etifoxine in the form of R-Etifoxine for use in the treatment of a disease related to activated mast cells, wherein R-Etifoxin is more potent than S-Etifoxine.

In another preferred embodiment, the present disclosure relates to Etifoxine in the form of S-Etifoxine for use in the treatment of a disease related to activated mast cells, wherein the treatment comprises oral, parenteral, subcutaneous, intravenous, mucosal, intracutaneous, intramuscular, intraperitoneal, transdermal, intranasal, transmucosal, rectal, sublingual, intravaginal, gastrointestinal, topical, buccal administration, or administration by inhalation. In a more preferred embodiment, the present disclosure relates to Etifoxine in the form of S-Etifoxine for use in the treatment of a disease related to activated mast cells, wherein the treatment comprises oral, sublingual, buccal, parenteral, intravenous, subcutaneous, topical, intravaginal, transdermal administration, or administration by inhalation. In a most preferred embodiment, the present disclosure relates to Etifoxine in the form of S-Etifoxine for use in the treatment of a disease related to activated mast cells, wherein the treatment comprises oral, parenteral, topical, transdermal administration, or administration by inhalation.

In another preferred embodiment, the present disclosure relates to Etifoxine in the form of S-Etifoxine for use in the treatment of a disease related to activated mast cells, wherein S-Etifoxin is more potent than R-Etifoxine.

In another preferred embodiment, the present disclosure relates to Etifoxine in the racemic form for use in the treatment of a disease related to activated mast cells, wherein the treatment comprises oral, parenteral, subcutaneous, intravenous, mucosal, intracutaneous, intramuscular, intraperitoneal, transdermal, intranasal, transmucosal, rectal, sublingual, intravaginal, gastrointestinal, topical, buccal administration, or administration by inhalation. In a more preferred embodiment, the present disclosure relates to Etifoxine in the racemic form for use in the treatment of a disease related to activated mast cells, wherein the treatment comprises oral, sublingual, buccal, parenteral, intravenous, subcutaneous, topical, intravaginal, transdermal administration, or administration by inhalation. In a most preferred embodiment, the present disclosure relates to Etifoxine in the racemic form for use in the treatment of a disease related to activated mast cells, wherein the treatment comprises oral, parenteral, topical, transdermal administration, or administration by inhalation.

### Pharmaceutical Composition for Use in the Treatment of a Disease Related to Mast Cell Activation

In another embodiment, the present disclosure relates to a pharmaceutical composition comprising Etifoxine and a pharmaceutically acceptable excipient for use in the treatment of a disease related to activated mast cells. As used herein, the term “pharmaceutical composition” refers to a “pharmaceutical composition comprising Etifoxine, or a pharmaceutically acceptable derivative thereof, and a pharmaceutically acceptable excipient”.

In a preferred embodiment, Etifoxine is present in the pharmaceutical composition as S-Etifoxine, R-Etifoxine, or a mixture thereof (e.g., racemate).

In a preferred embodiment, the present disclosure relates to a pharmaceutical composition for use in the treatment of a disease related to activated mast cells, wherein the disease is selected from the group consisting of asthma, allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, H1N1 influenza, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, tumor growth, metastasis, cancer, autoimmune disorders, inflammatory disorders, and fibrotic disorders, preferably wherein the disease is selected from the group consisting of allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, autoimmune disorders, inflammatory disorders, and fibrotic disorders.

In another embodiment, the present disclosure relates to a pharmaceutical composition for use in the treatment of a disease related to activated mast cells, wherein the disease is selected from the group consisting of viral hemorrhagic fever caused by a virus, wherein the virus is a member of at least one of the virus families Arenaviridae, Filoviridae, Bunyaviridae, Flaviviridae, and Rhabdoviridae. In a preferred embodiment, the virus comprises Dengue virus, Ebola virus, Marburg virus, Hanta virus, West Nile virus, Saint Louis encephalitis, Kunjin virus, Lassa virus, Crimean-Congo hemorrhagic fever virus, Rift Valley fever virus, yellow fever virus, Japanese encephalitis virus, Lujo virus, Junin virus, Argentine hemorrhagic fever virus, Brazilian hemorrhagic fever virus, Bolivian hemorrhagic fever virus, Argentine hemorrhagic fever virus, Garissa virus, SFTS virus, Hantan virus, Omsk hemorrhagic fever virus, Kyasanur forest disease virus, Langat virus, Alkhurma virus, or Henipa virus.

In another embodiment, the present disclosure relates to a pharmaceutical composition for use in the treatment of a disease related to activated mast cells, wherein the disease is selected from the group consisting of viral infections, wherein the virus is a member of at least one virus family selected from the group consisting of Coronaviridae and Orthomyxoviridae. In a preferred embodiment, the virus comprises MERS-CoV, SARS-CoV, SARS-CoV-2, influenza A, or H1N1 influenza.

In a preferred embodiment, the present disclosure relates to a pharmaceutical composition for use in the treatment of a disease related to activated mast cells, wherein the disease is selected from the group consisting of allergy, asthma, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, tumor growth, metastasis, cancer, fibrotic disorders, and autoimmune disorders, preferably wherein the disease is selected from the group consisting of allergy, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, fibrotic disorders, and autoimmune disorders.

In another preferred embodiment, the present disclosure relates to a pharmaceutical composition for use in the treatment of a disease related to activated mast cells, wherein the disease is selected from the group consisting of viral hemorrhagic fever caused by a virus comprising Dengue virus, West Nile virus, Crimean-Congo hemorrhagic fever virus, or Japanese encephalitis virus.

In another preferred embodiment, the present disclosure relates to a pharmaceutical composition for use in the treatment of a disease related to activated mast cells, wherein the disease is selected from the group consisting of viral infection caused by a virus comprising SARS-CoV-2 or H1N1 influenza.

In yet another preferred embodiment, the present disclosure relates to a pharmaceutical composition for use in the treatment of a disease related to activated mast cells, wherein the disease is selected from the group consisting of allergy, asthma, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, systemic mast cell activation disease, fibrotic disorders, tumor growth, metastasis, and cancer, preferably wherein the disease is selected from the group consisting of allergy, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, systemic mast cell activation disease and fibrotic disorders.

In a more preferred embodiment, the present disclosure relates to a pharmaceutical composition for use in the treatment of a disease related to activated mast cells, wherein the disease is selected from the group consisting of allergy, asthma, autoimmune disorders, chronic fatigue syndrome, chronic lyme disease, fibromyalgia, fibrotic disorders, inflammatory bowel disease, irritable bowel syndrome, systemic mastocytosis, and systemic mast cell activation disease, preferably wherein the disease is selected from the group consisting of allergy, autoimmune disorders, chronic fatigue syndrome, chronic lyme disease, fibromyalgia, fibrotic disorders, inflammatory bowel disease, irritable bowel syndrome, systemic mastocytosis, and systemic mast cell activation disease.

In another more preferred embodiment, the present disclosure relates to a pharmaceutical composition for use in the treatment of a disease related to activated mast cells, wherein the disease is selected from the group consisting of viral hemorrhagic fever caused by Dengue virus.

In another more preferred embodiment, the present disclosure relates to a pharmaceutical composition for use in the treatment of a disease related to activated mast cells, wherein the disease is selected from the group consisting of viral infections caused by SARS-CoV-2.

In a most preferred embodiment, the present disclosure relates to a pharmaceutical composition for use in the treatment of a disease related to activated mast cells, wherein the disease is selected from the group consisting of allergy, fibromyalgia, fibrotic disorders, inflammatory bowel disease, irritable bowel syndrome, systemic mastocytosis, and systemic mast cell activation disease.

In another most preferred embodiment, the present disclosure relates to a pharmaceutical composition for use in the treatment of a disease related to activated mast cells, wherein the disease is selected from the group consisting of viral hemorrhagic fever caused by Dengue virus.

In another most preferred embodiment, the present invention relates to a pharmaceutical composition for use in the treatment of a disease related to activated mast cells, wherein the disease is selected from the group consisting of allergy, atherosclerosis, bladder pain syndrome, chronic fatigue syndrome, chronic lyme disease, colitis ulcerosa, Dengue fever, fibromyalgia, fibrotic disease, interstitial cystitis, irritable bowel syndrome, obesity, SARS-CoV-2 (Covid-19), systemic mast cell activation syndrome, systemic lupus erythematosus, thrombotic disease, urticarial, viral infections and vulvodynia.

In another most preferred embodiment, the present disclosure relates to a pharmaceutical composition for use in the treatment of a disease related to activated mast cells, wherein the disease is selected from the group consisting of viral infections caused by SARS-CoV-2.

In another preferred embodiment, the present disclosure relates to a pharmaceutical composition for use in the treatment of a disease related to activated mast cells, wherein the pharmaceutical composition further comprises at least one compound selected from the group consisting of binder, filler, diluent, disintegrating agent, lubricant, glidant, flavouring agent, wetting agent, emulsifying agent, pH buffering agent, sweetening agent, and a mixture thereof. In a preferred embodiment, the pharmaceutical composition further comprises starch, glucose, lactose, sucrose, gelatin, malt, rice, malt, flour, silica gel, sodium stearate, glycerol monostearate, talc, sodium chloride, dried skim milk, glycerol, propylene, glycol, water, or a mixture thereof.

In another preferred embodiment, the present disclosure relates to a pharmaceutical composition for use in the treatment of a disease related to activated mast cells, wherein the treatment comprises oral, parenteral, subcutaneous, intravenous, mucosal, intracutaneous, intramuscular, intraperitoneal, transdermal, intranasal, transmucosal, rectal, sublingual, intravaginal, gastrointestinal, topical, buccal administration, or administration by inhalation. In a more preferred embodiment, the present disclosure relates to a pharmaceutical composition for use in the treatment of a disease related to activated mast cells, wherein the treatment comprises oral, sublingual, buccal, parenteral, intravenous, subcutaneous, topical, intravaginal, transdermal administration, or administration by inhalation. In a most preferred embodiment, the present disclosure relates to a pharmaceutical composition for use in the treatment of a disease related to activated mast cells, wherein the treatment comprises oral, parenteral, topical, transdermal administration, administration or by inhalation. The route of administration is left to the discretion of the practitioner. In most instances, administration will result in the release of Etifoxine into the bloodstream.

In another embodiment, Etifoxine can be delivered in a vesicle, in particular a liposome (see Langer, “New Methods of Drug Delivery,” Science 249:1527-1533 (1990); Lopez-Berestein, “Treatment of Systemic Fungal Infections with Liposomal-Amphotericin B,” Liposomes in the Therapy of Infectious Disease and Cancer, pp. 317-327 (1989); and Treat et al., “Liposome encapsulated doxorubicin—preliminary results of phase I and phase II trials” Liposomes in the Therapy of Infectious Disease and Cancer, pp. 353-365 (1989)).

In another embodiment, Etifoxine can be delivered in a controlled-release system or sustained-release system (see, e.g., Goodson, “Dental Applications,” pp. 115-138 in Medical Applications of Controlled Release, Vol. 2, Applications and Evaluation, Langer and Wise, eds., CRC Press (1984), hereafter “Goodson”). Other controlled- or sustained-release systems discussed in the review by Langer, Science 249:1527-1533 (1990) can be used. In one embodiment, a pump can be used (Langer, Science 249:1527-1533 (1990); Sefton, “Implantable Pumps,” in CRC Crit. Rev. Biomed. Eng. 14(3):201-240 (1987); Buchwald et al., “Long-term, Continuous Intravenous Heparin Administration by an Implantable Infusion Pump in Ambulatory Patients with Recurrent Venous Thrombosis,” Surgery 88:507-516 (1980); and Saudek et al., “A Preliminary Trial of the Programmable Implantable Medication System for Insulin Delivery,” New Engl. J. Med. 321:574-579 (1989)). In another embodiment, polymeric materials can be used (see Goodson; Smolen et al., “Drug Product Design and Performance,” Controlled Drug Bioavailability Vol. 1, John Wiley & Sons, New York (1984); Langer et al., “Chemical and Physical Structure of Polymers as Carriers for Controlled Release of Bioactive Agents: A Review,” J. Macromol. Sci. Rev. Macromol. Chem. C23(1):61-126 (1983); Levy et al., “Inhibition of Calcification of Bioprosthetic Heart Valves by Local Controlled-Release Diphosphonate,” Science 228:190-192 (1985); During et al., “Controlled Release of Dopamine from a Polymeric Brain Implant: In Vivo Characterization,” Ann. Neurol. 25:351-356 (1989); and Howard et al., “Intracerebral drug delivery in rats with lesion-induced memory deficits,” J. Neurosurg. 71:105 (1989)). In yet another embodiment, a controlled- or sustained-release system can be placed in proximity of a target of Etifoxine thus requiring only a fraction of the systemic dose.

The pharmaceutical composition can optionally comprise a suitable amount of a pharmaceutically acceptable excipient so as to provide the form for proper administration to the subject. Such a pharmaceutical excipient can be a diluent, suspending agent, solubilizer, binder, disintegrant, preservative, coloring agent, lubricant, and the like. The pharmaceutical excipient can be a liquid, such as water or an oil, including those of petroleum, animal, vegetable, or synthetic origin, such as peanut oil, soybean oil, mineral oil, sesame oil, and the like. The pharmaceutical excipient can be saline, gum acacia, gelatin, starch paste, talc, keratin, colloidal silica, urea, and the like. In addition, auxiliary, stabilizing, thickening, lubricating, and coloring agents can be used. In one embodiment, the pharmaceutically acceptable excipient is sterile when administered to a subject. Water is a particularly useful excipient when Etifoxine is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The compositions, if desired, can also contain minor amounts of wetting or emulsifying agents, or pH buffering agents. Specific examples of pharmaceutically acceptable carriers and excipients that can be used to formulate oral dosage forms are described in the Handbook of Pharmaceutical Excipients, (Amer. Pharmaceutical Ass'n, Washington, D C, 1986), incorporated herein by reference.

The pharmaceutical compositions can take the form of solutions, suspensions, emulsion, tablets, pills, pellets, multiparticulates, capsules, capsules containing liquids, powders, multiparticulates, sustained-release formulations, suppositories, emulsions, aerosols, sprays, suspensions, or any other form suitable for use. In one embodiment, the pharmaceutical composition is in the form of a capsule (see e.g., U.S. Pat. No. 5,698,155). Other examples of suitable pharmaceutical excipients are described by Radebough et al., “Preformulation,” pp. 1447-1676 in Remington's Pharmaceutical Sciences Vol. 2 (Gennaro, ed., 19th ed., Mack Publishing, Easton, Pa., 1995).

In one embodiment, Etifoxine is formulated in accordance with routine procedures as a composition adapted for oral administration to human beings. Etifoxine to be orally delivered can be in the form of tablets, capsules, gelcaps, caplets, lozenges, aqueous or oily solutions, suspensions, granules, powders, emulsions, syrups, or elixirs, for example. When Etifoxine is incorporated into oral tablets, such tablets can be compressed, tablet triturates, enteric-coated, sugar-coated, film-coated, multiply compressed or multiply layered. Techniques and compositions for making solid oral dosage forms are described in Pharmaceutical Dosage Forms: Tablets (Lieberman et al., eds., 2nd ed., Marcel Dekker, Inc., 1989 & 1990). Techniques and compositions for making tablets (compressed and molded), capsules (hard and soft gelatin) and pills are also described by King, “Tablets, Capsules, and Pills,” pp. 1553-1593 in Remington's Pharmaceutical Sciences (Osol, ed., 16th ed., Mack Publishing, Easton, Pa., 1980).

Liquid oral dosage forms include aqueous and nonaqueous solutions, emulsions, suspensions, and solutions and/or suspensions reconstituted from non-effervescent granules, optionally containing one or more suitable solvents, preservatives, emulsifying agents, suspending agents, diluents, sweeteners, coloring agents, flavoring agents, and the like. Techniques and composition for making liquid oral dosage forms are described in Pharmaceutical Dosage Forms: Disperse Systems (Lieberman et al., eds., 2nd ed., Marcel Dekker, Inc., 1996 & 1998).

Orally administered Etifoxine can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

When Etifoxine is to be injected parenterally, it can be, e.g., in the form of an isotonic sterile solution. Alternatively, when Etifoxine is to be inhaled, it can be formulated into a dry aerosol or can be formulated into an aqueous or partially aqueous solution. When Etifoxine is to be administered topically, it can be formulated into a creme, ointment, gel, for example. When Etifoxine is to be administered transdermally, it can be formulated into a transdermal patch, for example.

In another embodiment, Etifoxine can be formulated for intravenous administration. In one embodiment, compositions for intravenous administration comprise sterile isotonic aqueous buffer. Where necessary, the compositions can also include a solubilizing agent. Etifoxine for intravenous administration can optionally include a local anesthetic such as benzocaine or prilocaine to lessen pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where Etifoxine is to be administered by infusion, it can be dispensed, for example, with an infusion bottle containing sterile pharmaceutical grade water or saline. Where Etifoxine is administered by injection, an ampule of sterile water for injection or saline can be provided so that the ingredients can be mixed prior to administration.

Etifoxine can be administered by controlled-release or sustained-release means or by delivery devices that are known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566. Such dosage forms can be used to provide controlled- or sustained-release of one or more active ingredients using, for example, hydropropylmethyl cellulose, ethylcellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those of ordinary skill in the art, including those described herein, can be readily selected for use with the active ingredients of the disclosure. The disclosure thus encompasses single unit dosage forms suitable for oral administration such as, but not limited to, tablets, capsules, gelcaps, and caplets that are adapted for controlled- or sustained-release.

Controlled- or sustained-release pharmaceutical compositions can have a common goal of improving drug therapy over that achieved by their non-controlled or non-sustained release counterparts. In one embodiment, a controlled- or sustained-release composition comprises a minimal amount of Etifoxine to cure or control the condition in a minimum amount of time. Advantages of controlled- or sustained-release compositions include extended activity of the drug, reduced dosage frequency, and increased patient compliance. In addition, controlled- or sustained-release compositions can favorably affect the time of onset of action or other characteristics, such as blood levels of Etifoxine and can thus reduce the occurrence of adverse side effects.

Controlled- or sustained-release compositions can be designed to immediately release an amount of Etifoxine or a pharmaceutically acceptable derivative thereof, that promptly produces the desired therapeutic or prophylactic effect, and gradually and continually release other amounts of Etifoxine to maintain this level of therapeutic or prophylactic effect over an extended period of time. To maintain a constant level of Etifoxine in the body, the Etifoxine can be released from the dosage form at a rate that will replace the amount of Etifoxine being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of Etifoxine or a pharmaceutically acceptable derivative thereof, that is effective in the treatment or prevention of a disorder can be determined by standard clinical techniques. In addition, in vitro or in vivo assays can optionally be employed to help identify optimal dosage ranges. The precise dose to be employed will also depend on the route of administration, and the seriousness of the disorder and can be decided according to the judgment of a practitioner and/or each subject's circumstances.

In one embodiment, an effective dosage amount of Etifoxine is administered about every 96 h until the disorder is abated. In another embodiment, an effective dosage amount is administered about every 72 h until the disorder is abated. In another embodiment, an effective dosage amount is administered about every 24 h until the disorder is abated. In another embodiment, an effective dosage amount is administered about every 12 h until the disorder is abated. In another embodiment, an effective dosage amount is administered about every 8 h until the disorder is abated. In another embodiment, an effective dosage amount is administered about every 6 h until the disorder is abated. In another embodiment, an effective dosage amount is administered about every 4 h until the disorder is abated. In another embodiment, an effective dosage amount is administered about every 3 h until the disorder is abated.

In another embodiment, effective dosage amounts of Etifoxine range from of 1 mg to 2000 mg/day, or from 5 mg to 1000 mg/day, or from 10 mg to 800 mg/day, or from 13 mg to 600 mg/day, or from 15 mg to 500 mg/day, or from 17 mg to 400 mg/day, or from 20 mg to 300 mg/day, or from 25 mg to 250 mg/day, or from 30 mg to 200 mg/day, or from 35 mg to 150 mg/day, or from 40 mg to 100 mg/day, 45 mg to 85 mg/day or from 50 mg to 70 mg/day.

The methods for treating or preventing a disorder in a subject identified as in need thereof can further comprise administering to the subject Etifoxine (i.e., a first therapeutic agent), another therapeutic agent (i.e., a second therapeutic agent, another therapeutic drug, or a second therapeutic drug).

The second therapeutic agent can be, but is not limited to, an anticancer drug, a non-opioid analgesic, a pro-apoptotic agent, a non-steroid anti-inflammatory agent, an antimigraine agent, a Cox-II inhibitor, an antiemetic, an anticonvulsant, an antidepressant, a Ca²⁺-channel blocker, an agent for treating or preventing an autoimmune disorder and/or an inflammatory disorder, an agent for treating or preventing fibrotic disorders, an agent for treating addictive disorder, an agent for treating Parkinson's disease and parkinsonism, an agent for treating anxiety, an agent for treating epilepsy, an agent for treating a stroke, an agent for treating a seizure, an agent for treating a pruritic condition, an agent for treating psychosis, an agent for treating a cognitive disorder, an agent for treating a migraine, an agent for treating vomiting, an agent for treating dyskinesia, an agent for treating depression, a pharmaceutically acceptable derivative thereof, or any mixture thereof.

Examples of useful pro-apoptotic agents include, but are not limited thereto, BCL2 inhibitors, BH₃-mimetics, IAP inhibitors, MDM2 inhibitors, proteasome inhibitors, obatoclax, bortezomib, venetoclax, brentuximab vedotin, trastuzumab emtansone, gemtuzumab ozogamicine, cytotoxic and cytostatic agents such as alkylating agents, anthracyclines (such as idarubicin), cytoskeletal disruptors (also referred to as “taxanes”), epothilones, histone deacetylase inhibitors, inhibitors of topoisomerase I, inhibitors of topoisomerase II, nucleotide analogs and precursors, analogs, peptide antibiotics, platinum-based agents, retiniods, vinca alkaloids and derivatives, lysosomotropic agents (such as mefloquine), a pharmaceutically acceptable derivative thereof, or any mixture thereof.

Examples of useful non-opioid analgesics include, but are not limited to, non-steroidal analgesic and antipyretic agents, such as acetaminophen and metamizol.

Examples of useful antimigraine agents include, but are not limited to, alpiropride, bromocriptine, dihydroergotamine, dolasetron, ergocornine, ergocorninine, ergocryptine, ergonovine, ergot, ergotamine, flumedroxone acetate, fonazine, ketanserin, lisuride, lomerizine, methylergonovine, methysergide, naratriptan, oxetorone, pizotyline, risperidone, rizatriptan, sumatriptan, trazodone, zolmitriptan, a pharmaceutically acceptable derivative thereof, or any mixture thereof.

Examples of useful Cox-II inhibitors and 5-lipoxygenase inhibitors, as well as combinations thereof, are described in U.S. Pat. No. 6,136,839. Examples of useful Cox-II inhibitors include, but are not limited to, celecoxib, DUP-697, flosulide, meloxicam, 6-MNA, L-745337, rofecoxib, nabumetone, nimesulide, NS-398, SC-5766, T-614, L-768277, GR-253035, JTE-522, RS-57067-000, SC-58125, SC-078, PD-138387, NS-398, flosulide, D-1367, SC-5766, PD-164387, etoricoxib, valdecoxib, parecoxib, a pharmaceutically acceptable derivative thereof, or any mixture thereof.

The second therapeutic agent can also be an agent useful for reducing any potential side effects of Etifoxine (e.g., drowsiness, allergic reactions such as skin rash, or nausea). For example, the second therapeutic agent can be an antiemetic agent. Examples of useful antiemetic agents include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, a pharmaceutically acceptable derivative thereof, or any mixture thereof.

Examples of useful anticonvulsants include, but are not limited to, acetylpheneturide, albutoin, aloxidone, aminoglutethimide, 4-amino-3-hydroxybutyric acid, atrolactamide, beclamide, buramate, calcium bromide, cinromide, clomethiazole, clonazepam, decimemide, diethadione, dimethadione, doxenitroin, eterobarb, ethadione, ethosuximide, ethotoin, felbamate, fluoresone, gabapentin, 5-hydroxytryptophan, lamotrigine, magnesium bromide, magnesium sulfate, mephenytoin, mephobarbital, metharbital, methetoin, methsuximide, 5-methyl-5-(3-phenanthryl)-hydantoin, 3-methyl-5-phenylhydantoin, narcobarbital, nimetazepam, nitrazepam, oxcarbazepine, paramethadione, phenacemide, phenetharbital, pheneturide, phenobarbital, phensuximide, phenylmethylbarbituric acid, phenytoin, phethenylate sodium, potassium bromide, pregabaline, primidone, progabide, sodium bromide, solanum, strontium bromide, suclofenide, sulthiame, tetrantoin, tiagabine, topiramate, trimethadione, valproic acid, valpromide, vigabatrin, zonisamide, a pharmaceutically acceptable derivative thereof, or any mixture thereof.

Examples of useful antidepressants include, but are not limited to, binedaline, caroxazone, citalopram, (S)-citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, zimeldine, a pharmaceutically acceptable derivative thereof, or any mixture thereof.

Examples of useful Ca²⁺-channel blockers include, but are not limited to, bepridil, clentiazem, diltiazem, fendiline, gallopamil, mibefradil, prenylamine, semotiadil, terodiline, verapamil, amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, fantofarone, perhexiline, a pharmaceutically acceptable derivative thereof, or any mixture thereof.

Etifoxine may further be combined with anticancer drugs. Examples of useful anticancer drugs include, but are not limited to, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II or rIL2), interferon alpha-2a, interferon alpha-2b, interferon alpha-n1, interferon alpha-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, compounds of the class of taxane, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride, a pharmaceutically acceptable derivative thereof, or any mixture thereof.

Examples of other anticancer drugs include, but are not limited to, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cetuximab; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dihydrotaxol; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; EGFR inhibitors, elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gefitinib; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; 4-ipomeanol; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; MEK inhibitor; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer drug; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; ruthenium compounds; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, a pharmaceutically acceptable derivative thereof, or any mixture thereof.

Examples of useful therapeutic agents for treating or preventing autoimmune disorders. allergic disorders or inflamatory disorders include, but are not limited to, inhibitors of TNF-alpha; inhibitors of IL-5; inhibitors of IL5Ralpha; inhibitors of IL4 and IL-13; inhibitors of JAK1/2/3; inhibitors of siglec-8; anti-IgE antibodies or inhibitors of IgE; inhibitors of IL-15; inhibitors of MICA; inhibitors of MICB; inhibitors of ULBP-1; inhibitors of ULBP-2; inhibitor of ULBP-3; inhibitors of IL-10, a pharmaceutically acceptable derivative thereof, or any mixture thereof.

Examples of useful therapeutic agents for treating or preventing a fibrotic disorder include, but are not limited to, antagonists of CCR2; inhibitors of TGF-β type 1 receptor; inhibitors of Smad3; inhibitors of tyrosine kinases, such as Imatinib or Nintedanib; inhibitors of HMG-CoA reductase, a pharmaceutically acceptable derivative thereof, or any mixture thereof. [Rosenbloo, J., et al., Biochimica et Biophysica Acta, 2012, 1832, 1088].

Examples of useful therapeutic agents for treating or preventing anxiety include, but are not limited to, benzodiazepines, such as alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam, and triazolam; non-benzodiazepine agents, such as buspirone, gepirone, ipsapirone, tiospirone, zolpicone, zolpidem, and zaleplon; tranquilizers, such as barbituates, e.g., amobarbital, aprobarbital, butabarbital, butalbital, mephobarbital, methohexital, pentobarbital, phenobarbital, secobarbital, and thiopental; propanediol carbamates, such as meprobamate and tybamate; a pharmaceutically acceptable derivative thereof; or any mixture thereof.

Examples of useful therapeutic agents for treating or preventing epilepsy include, but are not limited to, ethosuximide, gabapentin, lamotrigine, phenobarbital, phenytoin, primidone, valproic acid, trimethadione, benzodiazepines, γ vinyl GABA, acetazolamide, felbamate, a pharmaceutically acceptable derivative thereof, or any mixture thereof.

Examples of useful therapeutic agents for treating or preventing stroke include, but are not limited to, anticoagulants such as heparin; agents that break up clots such as streptokinase or tissue plasminogen activator; agents that reduce swelling such as mannitol or corticosteroids; acetylsalicylic acid, a pharmaceutically acceptable derivative thereof, or any mixture thereof.

Examples of useful therapeutic agents for treating or preventing a seizure include, but are not limited to, carbamazepine; ethosuximide; gabapentin; lamotrignine; phenobarbital; phenytoin; primidone; valproic acid; trimethadione; bemzodiaepines; gabapentin; lamotrigine; γ-vinyl GABA; acetazolamide; felbamate, a pharmaceutically acceptable derivative thereof, or any mixture thereof.

Examples of useful therapeutic agents for treating or preventing a pruritic condition include, but are not limited to, naltrexone; nalmefene; danazol; tricyclics such as amitriptyline, imipramine, and doxepin; antidepressants such as those given below, menthol; camphor; phenol; pramoxine; capsaicin; tar; steroids; antihistamines; a pharmaceutically acceptable derivative thereof; or any mixture thereof.

Examples of useful therapeutic agents for treating or preventing psychosis include, but are not limited to, phenothiazines such as chlorpromazine hydrochloride, mesoridazine besylate, and thoridazine hydrochloride; thioxanthenes such as chloroprothixene and thiothixene hydrochloride; clozapine; risperidone; olanzapine; quetiapine; quetiapine fumarate; haloperidol; haloperidol decanoate; loxapine succinate; molindone hydrochloride; pimozide; ziprasidone; a pharmaceutically acceptable derivative thereof; or any mixture thereof.

Examples of useful therapeutic agents for treating or preventing a migraine include, but are not limited to, alpiropride, bromocriptine, dihydroergotamine, dolasetron, ergocornine, ergocorninine, ergocryptine, ergonovine, ergot, ergotamine, flumedroxone acetate, fonazine, ketanserin, lisuride, lomerizine, methylergonovine, methysergide, naratriptan, oxetorone, pizotyline, risperidone, rizatriptan, sumatriptan, trazodone, zolmitriptan, a pharmaceutically acceptable derivative thereof, or any mixture thereof.

Examples of useful therapeutic agents for treating or preventing vomiting include, but are not limited to, 5-HT3 receptor antagonists, such as ondansetron, dolasetron, granisetron, and tropisetron; dopamine receptor antagonists, such as prochlorperazine, thiethylperazine, chlorpromazin, metoclopramide, and domperidone; glucocorticoids, such as dexamethasone; benzodiazepines, such as lorazepam and alprazolam; a pharmaceutically acceptable derivative thereof; or any mixture thereof.

Examples of useful therapeutic agents for treating or preventing dyskinesia include, but are not limited to, reserpine; tetrabenazine, a pharmaceutically acceptable derivative thereof, or any mixture thereof.

Examples of useful therapeutic agents for treating or preventing depression include, but are not limited to, tricyclic antidepressants, such as amitryptyline, amoxapine, bupropion, clomipramine, desipramine, doxepin, imipramine, maprotilinr, nefazadone, nortriptyline, protriptyline, trazodone, trimipramine, and venlaflaxine; selective serotonin reuptake inhibitors, such as citalopram, (S)-citalopram, fluoxetine, fluvoxamine, paroxetine; and setraline; monoamine oxidase inhibitors, such as isocarboxazid, pargyline, phenelzine, and tranylcypromine; psychostimulants, such as dextroamphetamine and methylphenidate, a pharmaceutically acceptable derivative thereof, or any mixture thereof.

In one embodiment, the second therapeutic agent is administered in an effective amount.

An effective amount of the second therapeutic agent(s) will be known to those skilled the art depending on the agent. However, it is well within the skilled artisan's purview to determine the second therapeutic agent's optimal effective-amount range. Etifoxine and the second therapeutic agent combined can act either additively or synergistically to treat the same disorder, or they can act independently of each other such that Etifoxine treats or prevents a first disorder and the second therapeutic agent treats or prevents a second disorder, which can be the same as the first disorder or another disease. In one embodiment of the disclosure, where a second therapeutic agent is administered to a subject for treatment of a disorder (e.g., fibrotic disorder or cancer), the minimal effective amount of Etifoxine will be less than its minimal effective amount would be where the second therapeutic agent is not administered. In this embodiment, Etifoxine and the second therapeutic agent can act synergistically to treat or prevent a disorder. In one embodiment, Etifoxine is administered concurrently with a second therapeutic agent as a single composition comprising an effective amount of Etifoxine and an effective amount of the second therapeutic agent. Alternatively, a composition comprising an effective amount of Etifoxine and a second composition comprising an effective amount of the second therapeutic agent are concurrently administered. In another embodiment, an effective amount of Etifoxine is administered prior or subsequent to administration of an effective amount of the second therapeutic agent. In this embodiment, Etifoxine is administered while the second therapeutic agent exerts its therapeutic effect, or the second therapeutic agent is administered while Etifoxine exerts its therapeutic effect for treating or preventing a disorder.

### In Vitro or Ex Vivo Methods of Inhibiting Mast Cell Activation

In another preferred embodiment, the disclosure provides an in vitro or ex vivo method of inhibiting mast cell activation comprising contacting the mast cell with Etifoxine. The method of inhibiting mast cell activation by Etifoxine can be carried out in vitro, for example, as an assay to select suitable stimulators of mast cell activation or to identify and quantify specific mediators degranulated by mast cells and inhibition of the release of specific mediators with an effective amount of Etifoxine. The method is also useful for inhibiting mast cell activation in a human tissue ex vivo by contacting the mast cells within the human tissue with an effective amount of Etifoxine.

In a preferred embodiment, the method is useful for treating diseases related to mast cell activation in a subject. In a preferred embodiment, the method is useful for treating asthma, allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, H1N1 influenza, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, tumor growth, metastasis, cancer, autoimmune disorders, inflammatory disorders, and fibrotic disorders, preferably the method is useful for treating allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, autoimmune disorders, inflammatory disorders, and fibrotic disorders.

In another preferred embodiment, the method is useful for treating viral hemorrhagic fever caused by a virus, wherein the virus is a member of at least one of the virus families Arenaviridae, Filoviridae, Bunyaviridae, Flaviviridae, and Rhabdoviridae. In a preferred embodiment, the virus comprises Dengue virus, Ebola virus, Marburg virus, Hanta virus, West Nile virus, Saint Louis encephalitis, Kunjin virus, Lassa virus, Crimean-Congo hemorrhagic fever virus, Rift Valley fever virus, yellow fever virus, Japanese encephalitis virus, Lujo virus, Junin virus, Argentine hemorrhagic fever virus, Brazilian hemorrhagic fever virus, Bolivian hemorrhagic fever virus, Argentine hemorrhagic fever virus, Garissa virus, SFTS virus, Hantan virus, Omsk hemorrhagic fever virus, Kyasanur forest disease virus, Langat virus, Alkhurma virus, or Henipa virus.

In another preferred embodiment, the method is useful for treating of viral infections, wherein the virus is a member of at least one virus family selected from the group consisting of Coronaviridae and Orthomyxoviridae. In a preferred embodiment, the virus comprises SARS-CoV-2, influenza A, or H1N1 influenza.

Etifoxine can be assayed in vitro, ex vivo or in vivo for the desired therapeutic activity prior to use in a subject. Animal model systems can be used to demonstrate safety and efficacy.

In a preferred embodiment, the in vitro or ex vivo method of inhibiting mast cell activation comprising contacting the mast cell with Etifoxine is associated with a dose-dependent reduction of degranulation, wherein the degranulation comprises release of pre-stored mediators. In a preferred embodiment, the in vitro or ex vivo method of inhibiting mast cell activation comprising contacting the mast cell with Etifoxine is associated with a dose-dependent reduction of degranulation, wherein the degranulation comprises release of de novo synthesized mediators. In another preferred embodiment, the in vitro or ex vivo method of inhibiting mast cell activation comprising contacting the mast cell with Etifoxine is associated with a dose-dependent reduction of degranulation, wherein the degranulation comprises release of pre-stored mediators and de novo synthesized mediators. In a more preferred embodiment, the in vitro or ex vivo method of inhibiting mast cell activation comprising contacting the mast cell with Etifoxine is associated with a dose-dependent reduction of degranulation, wherein the degranulation comprises release of biogenic amines, proteoglycans, proteases, tryptases, lysosomal enzymes, growth factors, phospholipid metabolites, cytokines, chemokines, peptides, or a mixture thereof.

In another preferred embodiment, the in vitro or ex vivo method of inhibiting mast cell activation comprising contacting the mast cell with Etifoxine is associated with a dose-dependent reduction of degranulation, wherein the degranulation comprises release of biogenic amines such as histamine, serotonin, or polyamines, proteoglycans such as heparin, chondroitin sulfates, or serglycin, proteases such as matrix metalloproteinases, chymase-1, cathepsin G, granzyme B, carboxypeptidase A3, or tryptases comprising tryptase-α, tryptase-βI, tryptase-βII, tryptase-βIII, tryptase-γ, and tryptase-δ, lysosomal enzymes such as glucuronidase, β-hexosaminidase, or arylsulfatase, growth factors such as GM-CSF, VEGF, NGF, or PDGF, phospholipid metabolites such as LTB₄, LTC₄, PGD₂, PGE₂, or PAF, cytokines such as bFGF, SCF, interferon α, interferon β, interferon γ, TNF-α, TGF-β, or interleukins comprising IL-1, IL-3, IL-4, IL-5, IL-6, IL-8, IL-9, IL-10, IL-12, IL-13, IL-14, IL-16, IL-17, IL-18, and IL-25, chemokines such as MIP-1α, MIP-1β, CXCL8, CCL2, CCL5, CCL7, CCL11, or CCL13, peptides such as CRH (corticotropin-releasing hormone), urocortin comprising urocortin, urocortin-2, urocortin-3, or VIP (vasoactive intestinal peptide), and/or further mediators such as peroxidase, phospholipase, renin, substance P, leptin, angiogenin, nitric oxide.

In a more preferred embodiment, the in vitro or ex vivo method of inhibiting mast cell activation comprising contacting the mast cell with Etifoxine is associated with a dose-dependent reduction of degranulation, wherein the degranulation comprises release of histamine, heparin, tryptases comprising tryptase-α, tryptase-βI, tryptase-βII, tryptase-βIII, tryptase-γ, and tryptase-δ, β-hexosaminidase, MIP-1α, MIP-1β, CXCL8, CCL2, CCL5, CCL7, CCL11, or CCL13, renin, substance P, CRH, VEGF, TNF-α, IL1-1, IL-6, IL-17, LTC₄, PGD₂, or a mixture thereof. In a most preferred embodiment, Etifoxine inhibits mast cell activation resulting in a decreased degranulation of mediators comprising histamine, heparin, tryptases comprising tryptase-α, tryptase-βI, tryptase-βII, tryptase-βIII, tryptase-γ, and tryptase-δ, β-hexosaminidase, MIP-1α, MIP-1β, CXCL8, CCL2, CCL5, CCL7, CCL11, or CCL13, L1-1, IL-6, IL-17, PGD₂, or a mixture thereof.

In a preferred embodiment, the in vitro or ex vivo method of inhibiting mast cell activation comprising contacting the mast cell with Etifoxine is associated with a dose-dependent reduction of degranulation, wherein the degranulation comprises release of β-hexosaminidase or IL-6 (see Example 1).

In another preferred embodiment, the in vitro or ex vivo method of inhibiting mast cell activation comprising contacting the mast cell with Etifoxine is associated with a dose-dependent reduction of degranulation, wherein the degranulation comprises release of tryptase (see Examples 2 to 6).

### EXPERIMENTAL SECTION

In the following, the disclosure is illustrated in more detail. However, it is understood that the scope of protection is only determined by the attached claims, not being restricted to any of the following Examples. The following Examples are set forth to assist in understanding the disclosure and should not be construed as specifically limiting the disclosure described and claimed herein. Such variations of the disclosure, including the substitution of all equivalents now known or later developed, that would be within the purview of those skilled in the art, and changes in formulation or changes in experimental design, are to be considered to fall within the scope of the disclosure incorporated therein.

Etifoxine (6-Chloro-2-N-ethyl-4-methyl-4-phenyl-4H-3,1-benzoxazine-2-amine) was purchased from Tocris Bioscience as a racematic mixture of R- and S-Etifoxine. The preparation of Etifoxine is within the routine work of a skilled person. One method to prepare Etifoxine is according to the synthetic procedures disclosed in WO 2007/109289 A1.

It has been surprisingly found that mast cell activation can be inhibited by Etifoxine, or a pharmaceutically acceptable derivative thereof. The Examples below demonstrate said efficacy of Etifoxine to inhibit the degranulation of mast cells by means of in vitro and ex vivo experiments.

### Biological Assays

Preparation of Murine Bone Marrow-Derived Mast Cells Preloaded with IgE:

According to the technique established by Razin et al. (Proc. Natl. Acad. Sci. USA 1981, 78:2559-2561), bone marrow cells (1×10⁶/ml) from 6 to 8 week old mice (129/Sv×C57BL/6) were cultured (37° C. and 5% CO₂) as single cell suspensions in RPMI 1640 medium (Sigma-Aldrich, Munich, Germany) supplemented with 15% fetal calf serum (FCS), 1% X63Ag8-653-conditioned medium (Karasuyama and Melchers, Eur. J. Immunol. 1988, 18:97-104), 2 mM L-glutamine, 10 μM β-mercaptoethanol, 50 units/ml penicillin, and 50 mg/ml streptomycin. At weekly intervals, the non-adherent cells were reseeded at 5×10⁵ cells/ml in fresh medium. After 4-6 weeks in culture, greater than 99% of the cells were KIT- and FIERI-positive as assessed by fluorescence-activated cell sorting (FACS) using phycoerythrin-labeled anti-KIT antibodies (Pharmingen, Mississauga, Canada) and fluorescein isothiocyanate (FITC)-labelled hamster anti-mouse FcεRIα antibodies (eBioscience, Frankfurt, Germany), respectively. The resulting cells are called bone marrow-derived mast cells (BMMCs).

For degranulation studies, BMMCs were preloaded with 0.15 μg/ml IgE anti-DNP (dinitrophenyl; Sigma Aldrich, Munich, Germany) overnight at 37° C. The cells were then washed and resuspended in Tyrode's buffer (130 mM NaCl, 5 mM KCl, 1.4 mM CaCl₂, 1 mM MgCl₂, 5.6 mM glucose, and 0.1% bovine serum albumin (BSA) in 10 mM Hepes, pH 7.4).

### In Vitro Inhibition of Activated Murine Mast Cells by Etifoxine and Determination of Reduced Release of β-Hexosaminidase:

10⁶ Cells/1 ml obtained according to the protocol described above were adapted to 37° C. for 20 min and then incubated at 37° C. with vehicle (DMSO, control; different volumes of DMSO were used corresponding to the volumes needed to prepare the different concentrations of Etifoxine) or Etifoxine (3 μM, 10 μM, and 30 μM; dissolved in DMSO if necessary) for 20 min. Subsequently, the cells were left unstimulated (unst) or stimulated with 20 ng/ml antigen (Ag: dinitrophenyl-human serum albumin ([DNP-HSA]; dissolved in medium); Sigma Aldrich, Munich, Germany) for 30 min. The degree of degranulation was determined by measuring the release of β-hexosaminidase (Nishizumi et al., J Immunol 1997; 158:2350-2355). Experiments were performed in triplicates.

### In Vitro Inhibition of Activated Murine Mast Cells by Etifoxine and Determination of Reduced Release of Interleukin-6 (IL-6):

10⁶ Cells/1 ml obtained according to the protocol described above were adapted to 37° C. for 20 min and then incubated at 37° C. with vehicle (DMSO, control; different amounts of DMSO were used corresponding to the amounts needed to prepare the different concentrations of Etifoxine) or Etifoxine (3 μM, 10 μM, and 30 μM; dissolved in DMSO if necessary) for 20 min. Subsequently, the cells were left unstimulated (unst) or stimulated with 20 ng/ml Ag (dissolved in medium) for 3 h. Subsequently, the cells were pelleted by centrifugation and the resulting cell pellet was discarded. For the determination of the amount of degranulated IL-6, the respective supernatant obtained by centrifugation was subjected to an IL-6 ELISA according to the manufacturer's instructions (BD, Heidelberg, Germany). Experiments were performed in triplicate.

### Preparation of Human Nasal Tissue Samples for Human Ex Vivo Model:

Fresh human nasal mucosa was used (middle nasal turbinate), which was surgically removed as standard procedure during nose correction surgeries (septorhinoplasty with conchotomy). Only tissues from those patients who had not previously received medication in the form of cortisone preparations or antihistamines were used. The removed tissue was cleansed immediately after the surgical removal with Epilife Medium (Thermo Fisher Scientific, No. MEPICFPRF500), referred to as “medium” in terms of ex vivo experiments, a chemically defined, serum-free full medium for epithelial cells and keratinocytes. The tissue was then cut into 12 pieces of equal size, cleansed again in the medium, and the exact fresh weight of the cut tissue pieces was determined for later normalization and then stored overnight in cold medium. The weight of the tissue pieces varied between 10 and 20 mg.

Ex Vivo Inhibition of Human Tissue Mast Cells Activated Via LPS with Etifoxine:

The human nasal tissue pieces obtained according to the protocol described above were incubated in the following three compositions for one hour with 1.25 ml medium at 37° C. and 5% CO₂ in 24-well plates:
i) Two negative controls with pure medium.
ii) The medium together with Etifoxine (dissolved in DMSO, if necessary) in six concentrations: 3 μM (no DMSO required), 10 μM (no DMSO required), 30 μM (with DMSO in the ratio Etifoxine:DMSO=1:900), and 60 μM (with DMSO in the ratio Etifoxine:DMSO=1:450).
iii) For solvent control, DMSO diluted with medium in two different concentrations, 1:900 and 1:450 corresponding to the amounts of DMSO in the Etifoxine concentrations of 30 μM and 60 μM, respectively (Comparative Examples 1 to 3).

After an one hour of pre-incubation with the respective compositions i), ii) or iii), 4 μg/ml lipopolysaccharide (LPS, Sigma-Aldrich GmbH, L4516) was added to each well—except for one well serving as the negative control—in order to induce tryptase degranulation. Then, 250 μl supernatant were taken from each well at the time points of 15 minutes, 60 minutes (one hour), 120 minutes (two hours), 240 minutes (four hours), 480 minutes (eight hours), and 1200 minutes (20 hours) after stimulation with LPS and the supernatants were directly frozen. Immediately after the removal of 250 μl supernatant, the volume in each well was increased to the original level and the original composition by adding 250 μl of the respective composition i), ii) and iii).

For the determination of the amount of degranulated tryptase, the frozen supernatants were thawed at 22° C. and then subjected to a tryptase ELISA Kit from Boster Biological Technology Co., Ltd (No. EZ0898, Assay Range: 156-10000 pg/ml, Sensitivity: <15 pg/ml) according to manufacturer's instructions, and the ELISA measurements were performed with the Multiskan Go instrument from Thermo Scientific and the SkanIt Re 4.1 software.

The statistical analysis was conducted in two steps. In a first step, the raw measurement values were normalized to 10 mg tissue. In a second step, a correction factor was introduced for all time points of supernatant removal to compensate for the 250 μl supernatant taken for analysis at each time and the subsequent re-addition of medium incl. factors. The statistical calculations were performed with Microsoft Excel 2010.

Ex Vivo Inhibition of Human Tissue Mast Cells Activated Via SCF with Etifoxine:

The human nasal tissue pieces obtained according to the protocol described above were incubated in the following three compositions for one hour with 1.25 ml medium at 37° C. and 5% CO₂ in 24-well plates:
i) Two negative controls with pure medium.
ii) The medium together with Etifoxine in seven concentrations: 1 μM, 3 μM, 10 μM, 30 μM, 60 μM, 150 μM, and 300 μM.

After an one hour of pre-incubation with the respective compositions i) or ii), 0.4 μg/ml stem cell growth factor (SCF, Peprotech) was added to each well—except for one well serving as the negative control—in order to induce tryptase degranulation. Then, 250 μl supernatant were taken from each well at the time points of 120 minutes (two hours), 240 minutes (four hours), 480 minutes (eight hours), and 1200 minutes (20 hours) after stimulation with SCF and the supernatants were directly frozen. Immediately after the removal of 250 μl supernatant, the volume in each well was increased to the original level and the original composition by adding 250 μl of the respective composition i), and ii).

For the determination of the amount of degranulated tryptase, the frozen supernatants were thawed at 22° C. and then subjected to a tryptase ELISA Kit from Boster Biological Technology Co., Ltd (No. EZ0898, Assay Range: 156-10000 pg/ml, Sensitivity: <15 pg/ml) according to manufacturer's instructions, and the ELISA measurements were performed with the Multiskan Go instrument from Thermo Scientific and the SkanIt Re 4.1 software.

The statistical analysis was conducted in two steps. In a first step, the raw measurement values were normalized to 10 mg tissue. In a second step, a correction factor was introduced for all time points of supernatant removal to compensate for the 250 μl supernatant taken for analysis at each time and the subsequent re-addition of medium incl. factors. The statistical calculations were performed with Microsoft Excel 2010.

### Determination of IC₅₀ Values of Etifoxine:

In order to determine the IC₅₀ values of Etifoxine the program Graphpad Prism 8 was applied. Logarithmic concentrations of Etifoxine were plotted against the correlating tryptase concentrations determined in the ex vivo inhibition experiment with activated human nasal tissue mast cells (see Examples 3 and 5). For the analysis, a non-linear regression (variable slope—4-parametric) was used. The program setting “Dose-Response-Inhibition” was applied for an automatic calculation, readout and display of the IC₅₀ values.

### Comparative Example 1

### Effect of DMSO on the Activation of Murine Mast Cells Determined by the Release of β-Hexosaminidase (Degranulation)

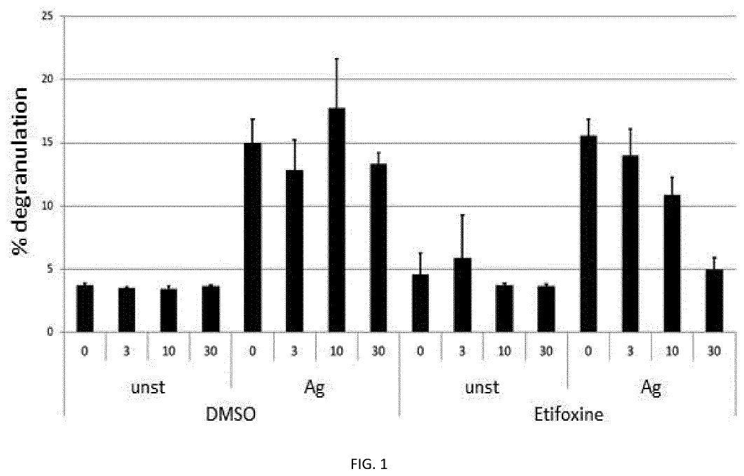

When unstimulated (in the absence of Ag), the activity of the IgE-loaded bone marrow-derived mast cells (BMMCs) was not influenced by the vehicle (DMSO) independent of its amount used, and was also not significantly affected by Etifoxine independently of the concentration studied (FIG. 1, left). In response to stimulation, DMSO also had no reducing effect on the degree of mast cell degranulation (determined by the minor effect of DMSO on the amount of β-hexosaminidase released).

### Comparative Example 2

### Effect of DMSO on the Activation of Murine Mast Cells Determined by the Release of Interleukin-6 (IL-6)

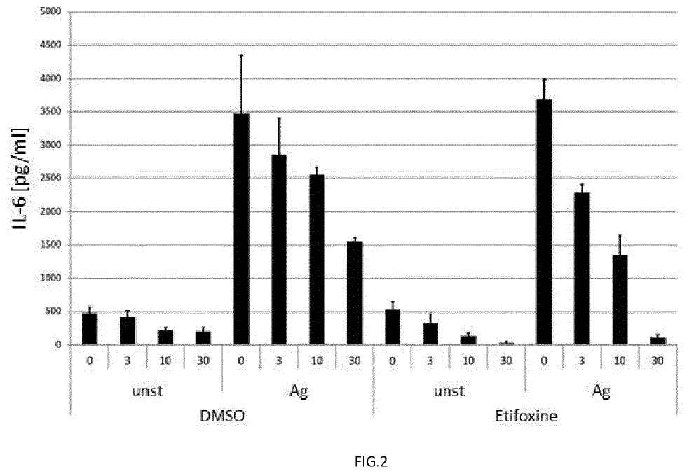

When unstimulated, mast cell degranulation of IL-6 was slightly attenuated with increasing amounts of DMSO (FIG. 2, left). Similarly, upon stimulation with Ag, DMSO induced a decrease of mast cell degranulation of IL-6 in a dose-dependent manner. This effect of DMSO on mast cells is well-known and therefore requires a thorough comparison of 30 μM Etifoxine dissolved in DMSO with the respective amounts of DMSO alone (see Example 2).

### Comparative Example 3

### Effect of DMSO on Degranulation of Activated Human Nasal Tissue Mast Cells

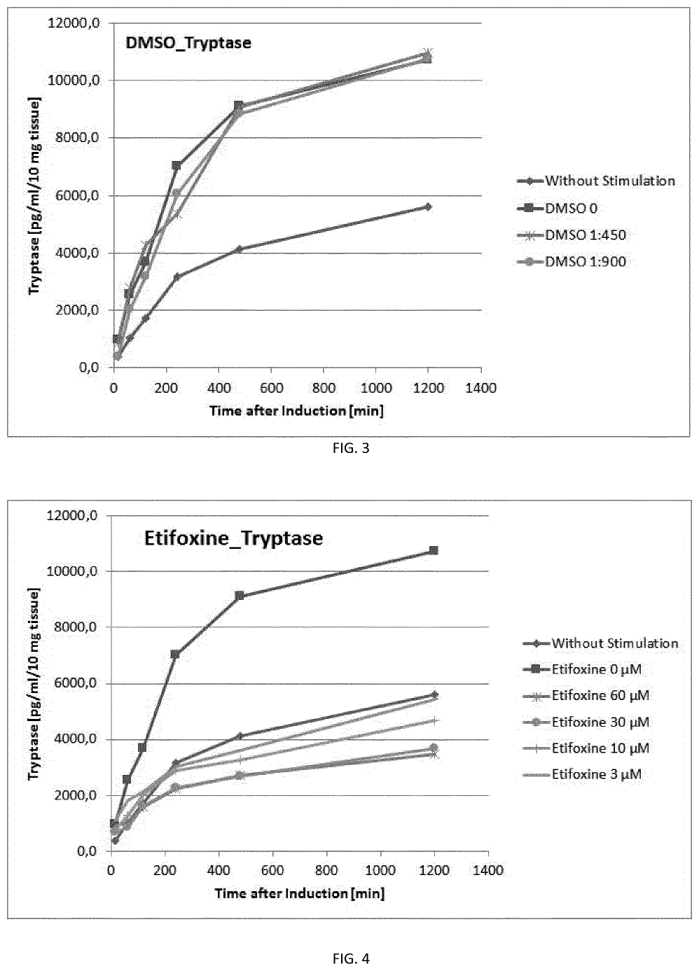

Increasing amounts of the solvent DMSO (as used in the respective ex vivo experiments using 30 μM or 60 μM of Etifoxin dissolved in DMSO, see Example 3) was shown to only slightly attenuate the LPS-stimulated degranulation of tryptase, and with decreasing effects over time (FIG. 3). The precisely measured values of said minor effect of DMSO on the degranulation of activated human nasal tissue mast cells are presented in Table 1.

TABLE 1
Minor effect of DMSO on the release of tryptase of human nasal
tissue mast cells activated with LPS.

| Time | Tryptase [pg/ml/10 mg tissue] | | DMSO:medium^{[1]} | |
|---|---|---|---|---|
| [min] | Without LPS | With LPS | 1:450^{[2]} | 1:900^{[3]} |
| 15 | 370.5 | 972.1 | 855.9 | 363.3 |
| 60 | 1026.4 | 2551.4 | 2803.9 | 2016.7 |
| 120 | 1708.0 | 3679.5 | 4268.2 | 3177.1 |
| 240 | 3174.1 | 7007.5 | 5363.7 | 6055.7 |
| 480 | 4136.4 | 9126.7 | 9082.6 | 8840.5 |
| 1200 | 5610.9 | 10747.9 | 10990.0 | 10762.3 |

^{[1]}With LPS.
^{[2]}Corresponding to the DMSO amount in 60 μM Etifoxine.
^{[3]}Corresponding to the DMSO amount in 30 μM Etifoxine.

### Example 1

### In Vitro Inhibition of Activated Murine Mast Cells by Etifoxin Determined by the Reduced Release of β-Hexosaminidase

Etifoxine clearly suppressed Ag-triggered degranulation in a dose-dependent manner as determined by the significantly reduced amount of the mediator β-hexosaminidase released from the mast cells (FIG. 1, right). The percentage of inhibition can be calculated to be 10% for 3 μM Etifoxine, 30% for 10 μM Etifoxine, and 68% for 30 μM Etifoxine. Based on the considerably reduced degranulation of β-hexosaminidase of up to 68%, it is demonstrated that Etifoxine is an efficient inhibitor of FcεRI-mediated degranulation of mast cells in vitro.

### Example 2

### In Vitro Inhibition of Activated Murine Mast Cells by Etifoxin Determined by the Reduced Release of Interleukin-6 (IL-6)

Treatment of the activated BMMCs with different concentrations of Etifoxine (3 μM, 10 μM, and 30 μM) dose-dependently suppressed Ag-induced release of IL-6 (FIG. 2, right). Taking into account the non-preventable solvent (DMSO)-induced reduction of IL-6 release (see Comparative Example 2), the percentage of inhibition can be calculated to be 20% for 3 μM Etifoxine, 48% for 10 μM Etifoxine, and 92% for 30 μM Etifoxine. Based on the considerably reduced release of IL-6 of up to 92%, it is demonstrated that Etifoxine is an efficient inhibitor of FcεRI-mediated activation of mast cells in vitro.

### Example 3

### Ex Vivo Inhibition of LPS-Induced Degranulation with Etifoxine

Treatment of human nasal tissue pieces with different concentrations of Etifoxine (3 μM, 10 μM, 30 μM, and 60 μM) was demonstrated to effectively and dose-dependently suppress LPS-induced mast cell degranulation of tryptase (FIG. 4 and Table 2).

TABLE 2
Effect of Etifoxine on the release of tryptase of human nasal tissue
mast cells activated with LPS.

| | Tryptase [pg/ml/10 | | | | | |
|---|---|---|---|---|---|---|
| Time | mg tissue] | | Etifoxine^{[1]} | | | |
| [min] | Without LPS | With LPS | 60 μM | 30 μM | 10 μM | 3 μM |
| 15 | 370.5 | 972.1 | 881.1 | 676.0 | 763.6 | 1107.7 |
| 60 | 1026.4 | 2551.4 | 1054.5 | 857.5 | 1252.6 | 1822.9 |
| 120 | 1708.0 | 3679.5 | 1592.2 | 1619.4 | 2035.3 | 2116.7 |
| 240 | 3174.1 | 7007.5 | 2241.2 | 2276.4 | 2901.8 | 3032.7 |
| 480 | 4136.4 | 9126.7 | 2720.1 | 2678.2 | 3275.1 | 3622.0 |
| 1200 | 5610.9 | 10747.9 | 3472.1 | 3675.9 | 4687.0 | 5419.5 |

^{[1]}With LPS.

Taking into account the minor, but non-preventable solvent (DMSO)-induced reduction of tryptase secretion for 30 μM and 60 μM Etifoxine, respectively (see Comparative Example 3), the percentage inhibition of degranulation by Etifoxine can be derived from the values across 120 minutes and 1200 minutes. The percentage inhibition of degranulation by Etifoxine at 120 minutes is as follows:
79% for 3 μM Etifoxine
83% for 10 μM Etifoxine
105% for 30 μM Etifoxine
106% for 60 μM Etifoxine

The percentage inhibition of degranulation by Etifoxine at 240 minutes is as follows:
104% for 3 μM Etifoxine
107% for 10 μM Etifoxine
123% for 30 μM Etifoxine
124% for 60 μM Etifoxine

The ex vivo experiment shows that Etifoxine effectively inhibits LPS-triggered tryptase degranulation completely achieving 100% inhibition in mast cells of human nasal pieces.

### Example 4

### Determination of IC₅₀ Values of Etifoxine

The IC₅₀ values for the ex vivo inhibition of tryptase release (degranulation) with Etifoxine in human nasal tissue (see Example 3) were determined at 2 hours (120 minutes) and 4 hours (240 minutes) after LPS addition, respectively. The IC₅₀ values of 14.71 μM and 15.1 μM, respectively, indicate a high efficacy of Etifoxine to inhibit human mast cell activation within a living human tissue environment. Detailed data for the determination of the IC₅₀ values can be deduced from Table 3 and FIG. 5.

TABLE 3
Determination of IC₅₀ values for ex vivo inhibition of tryptase
degranulation with Etifoxine in human nasal tissue after LPS
addition (two hours and four hours, respectively).

| | Tryptase 2 h | Tryptase 4 h |
|---|---|---|
| log(inhibitor) vs. response- | Perfect fit | Perfect fit |
| Variable slope (four parameters) | | |
| Bottom | 2239 | 1590 |
| Top | 3034 | 2117 |
| LogIC50 | 1.168 | 1.179 |
| HillSlope | −4.183 | −4.101 |
| IC₅₀ | 14.71 μM | 15.1 μM |
| Span | 794.7 | 527 |

### Example 5

### Ex Vivo Inhibition of SCF-Induced Degranulation with Etifoxine

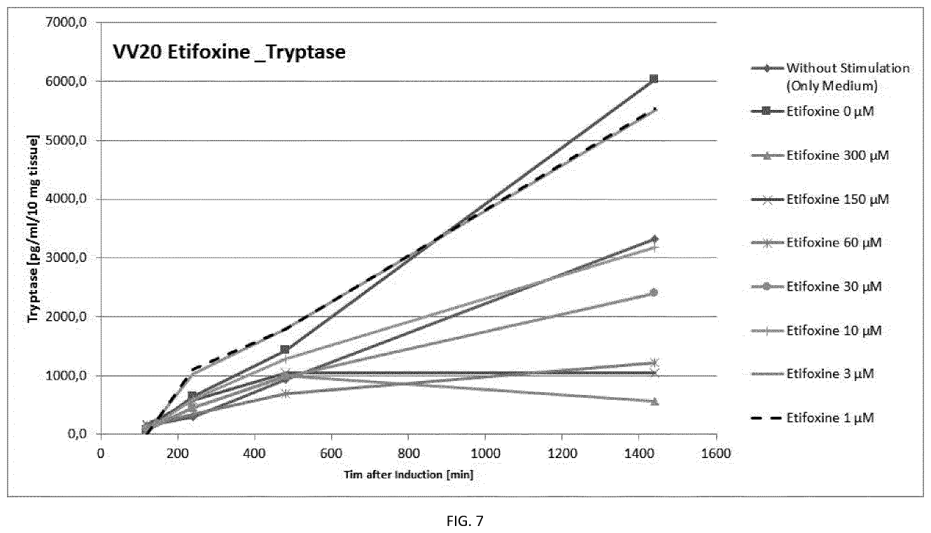

Treatment of human nasal tissue pieces with different concentrations of Etifoxine (1 μM, 3 μM, 10 μM, 30 μM, 60 μM, 150 μM, and 300 μM) was demonstrated to effectively and dose-dependently suppress SCF-induced mast cell degranulation of tryptase (FIG. 7 and Table 4).

The percentage inhibition of degranulation by Etifoxine can be derived from the values across 120 minutes and 1200 minutes. The percentage inhibition values at 240 minutes are as follows:
0% for 1 μM Etifoxine
0% for 3 μM Etifoxine
15% for 10 μM Etifoxine
56% for 30 μM Etifoxine
85% for 60 μM Etifoxine
21% for 150 μM Etifoxine
53% for 300 μM Etifoxine

The percentage inhibition of degranulation by Etifoxine at 480 minutes is as follows:
0% for 1 μM Etifoxine
0% for 3 μM Etifoxine
28% for 10 μM Etifoxine
90% for 30 μM Etifoxine
148% for 60 μM Etifoxine
77% for 150 μM Etifoxine
87% for 300 μM Etifoxine

The ex vivo experiment shows that Etifoxine effectively inhibits SCF-triggered tryptase release (degranulation) completely achieving 100% inhibition in mast cells of human nasal pieces.

TABLE 4
Effect of Etifoxine on the release of tryptase of human nasal tissue mast cells activated with SCF.

| | Tryptase [pg/ml/10 | | | | | | | | |
|---|---|---|---|---|---|---|---|---|---|
| | mg tissue] | | Etifoxine^{[1]} | | | | | | |
| Time [min] | Without SCF | With SCF | 300 μM | 150 μM | 60 μM | 30 μM | 10 μM | 3 μM | 1 μM |
| 120 | 154.2 | 66.1 | 71.9 | 163.3 | 152.7 | 109.4 | 71.9 | 38.7 | 12.1 |
| 240 | 290.8 | 651.6 | 459.3 | 576.8 | 345.9 | 449.9 | 594.5 | 1030.3 | 1113.7 |
| 480 | 928.4 | 1422.1 | 991.8 | 1042.4 | 689.7 | 979.7 | 1284.9 | 1789.0 | 1791.7 |
| 1440 | 3318.6 | 6031.6 | 559.4 | 1040.1 | 1218.2 | 2400.2 | 3185.9 | 5512.9 | 5547.4 |

^{[1]}With SCF.

### Example 6

### Determination of IC₅₀ Values of Etifoxine

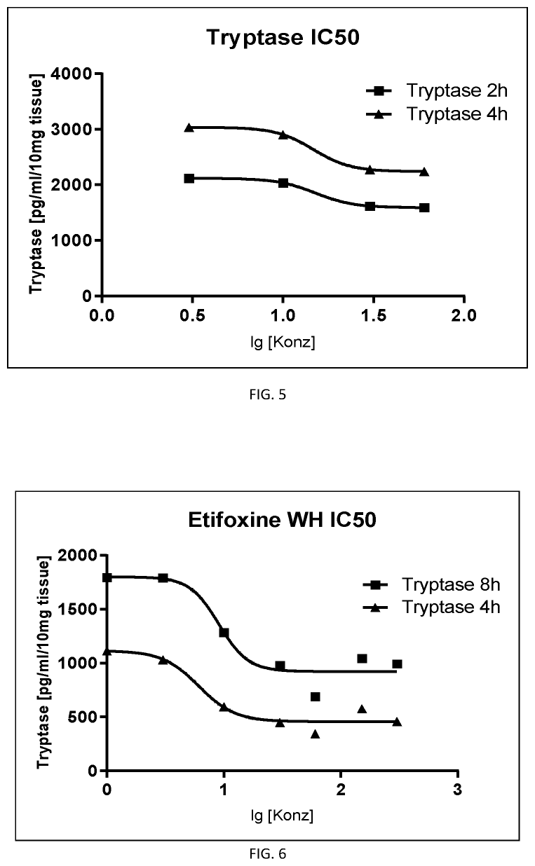

The IC₅₀ values for the ex vivo inhibition of tryptase degranulation with Etifoxine in human nasal tissue (see Example 3) were determined at 4 hours and 8 hours after SCF addition, respectively. The IC₅₀ values of 6.12 μM and 9.07 μM, respectively, indicate a high efficacy of Etifoxine to inhibit human mast cell activation within a living human tissue environment. Detailed data for the determination of the IC₅₀ values can be deduced from Table 5 and FIG. 6.

TABLE 5
Determination of IC₅₀ values for ex vivo inhibition of tryptase
degranulation with Etifoxine in human nasal tissue after SCF
addition (two hours and four hours, respectively).

| | Tryptase 8 h | Tryptase 4 h |
|---|---|---|
| log(inhibitor) vs. response- | Perfect fit | Perfect fit |
| Variable slope (four parameters) | | |
| Bottom | 922 | 456.7 |
| Top | 1799 | 1115 |
| LogIC50 | 0.9578 | 0.7865 |
| HillSlope | −3.492 | −2.791 |
| IC₅₀ | 9.07 μM | 6.12 μM |
| Span | 877.4 | 658.3 |

### Example 7

### Human In-Vivo Study

A human in-vivo clinical test was conducted treating a patient with therapy-resistant systemic mastocytosis with off-label oral application of Etifoxine in full consent and patient agreement. The results of the clinical test were closely monitored, captured and documented together with patient. The patient is male, 60 years old and diagnosed with systemic mastocytosis (WHO criteria, KIT816V positiv) with official diagnosis at the age of 35. The disease course over the past 25 year has been progressive and increasing therapy-resistant leading to job disability in the year 2019. The current daily treatment of the patient is: H1 antihistamine 3× daily, cromolyn 4×200 mg daily, PPI 110 mg daily, Vitamine C/Vitamine D upon need, Actonel, upon need decortin 40 to 60 mg. Xolair has been discontinued after 7 years upon onset of serum disease, and the patient was therapy-resistant towards the kinase-inhibitors Imatinib, Dasatinib and Sunitinib.

The patient was treated off-label with Etifoxine per oral application over a time-period of 6 weeks with an increasing dosage from 50 mg to 150 mg as follows:
Day 1 to 14=1×daily 50 mg (morning)
Day 14 to 28=2×daily 50 mg=100 mg (morning, noon)
Day 29 to 42=3×daily 50 mg=150 mg (morning, noon, evening)

The symptom intensity was determined by a visual analog score (VAS; 0=no symptoms, 10=excruciating symptom intensity) on a weekly basis starting with a VAS baseline value before starting the intake of Etifoxine.

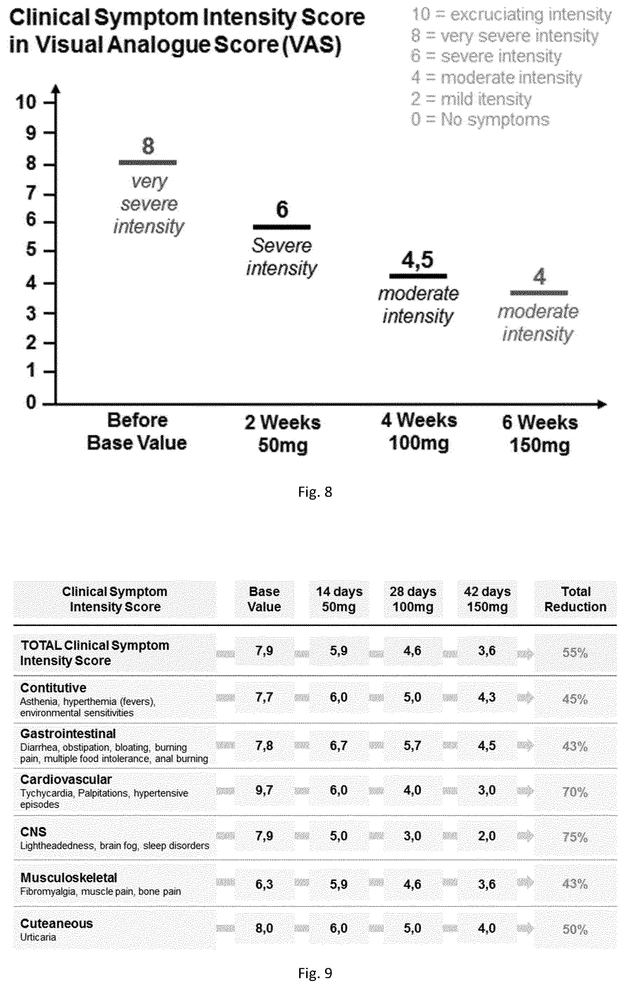

In overall the total clinical symptom intensity score resulted in a reduction of 50% over a timeframe of 6 weeks from a VAS baseline value of 8 to 4 after 6 weeks treatment with Etifoxine (see FIG. 8). No side effects occurred during the 6 week treatment with Etifoxine.

The reduction of clinical symptom intensity in the clinical symptom categories was significant in each category, however, varied in each clinical category (see FIG. 9):

The human off-label clinical test shows the capability of Etifoxine to inhibit mast cell activation and degranulation, and the potential for treatment of systemic mastocytosis and other diseases related to activated mast cells.

### Embodiments

The present application also pertains to the following numbered embodiments:
1. Etifoxine, or a pharmaceutically acceptable derivative thereof, for use in the treatment of a disease related to activated mast cells in a subject, preferably a human.
2. Etifoxine, or a pharmaceutically acceptable derivative thereof, for use according to claim 1, wherein the disease is selected from the group consisting of asthma, allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, H1N1 influenza, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, tumor growth, metastasis, cancer, autoimmune disorders, inflammatory disorders, fibrotic disorders, and viral hemorrhagic fever caused by a virus, wherein the virus is a member of at least one virus family selected from the group consisting of Arenaviridae, Filoviridae, Bunyaviridae, Flaviviridae, and Rhabdoviridae, preferably wherein the virus comprises Dengue virus, Ebola virus, Marburg virus, Hanta virus, West Nile virus, Saint Louis encephalitis, Kunjin virus, Lassa virus, Crimean-Congo hemorrhagic fever virus, Rift Valley fever virus, yellow fever virus, Japanese encephalitis virus, Lujo virus, Junin virus, Argentine hemorrhagic fever virus, Brazilian hemorrhagic fever virus, Bolivian hemorrhagic fever virus, Argentine hemorrhagic fever virus, Garissa virus, SFTS virus, Hantan virus, Omsk hemorrhagic fever virus, Kyasanur forest disease virus, Langat virus, Alkhurma virus, or Henipa virus.
3. Etifoxine, or a pharmaceutically acceptable derivative thereof, for use according to claim 1, wherein the disease is selected from the group consisting of allergy, asthma, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, tumor growth, metastasis, cancer, fibrotic disorders, autoimmune disorders, and viral hemorrhagic fever caused by a virus, wherein the virus comprises Dengue virus, West Nile virus, Crimean-Congo hemorrhagic fever virus, or Japanese encephalitis virus, preferably wherein the disease is selected from the group consisting of allergy, asthma, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, post-traumatic headache, systemic mast cell activation disease, fibrotic disorders, tumor growth, metastasis, cancer, and viral hemorrhagic fever caused by a virus, Dengue virus, West Nile virus, Crimean-Congo hemorrhagic fever virus, or Japanese encephalitis virus, more preferably wherein the disease is selected from the group consisting of allergy, asthma, autoimmune disorders, chronic fatigue syndrome, chronic lyme disease, fibromyalgia, fibrotic disorders, inflammatory bowel disease, irritable bowel syndrome, systemic mastocytosis, systemic mast cell activation disease, and viral hemorrhagic fever caused by a virus, Dengue virus, and most preferably wherein the disease is selected from the group consisting of allergy, asthma, fibromyalgia, fibrotic disorders, inflammatory bowel disease, irritable bowel syndrome, systemic mastocytosis, systemic mast cell activation disease, and viral hemorrhagic fever caused by a virus, Dengue virus.
4. Etifoxine, or a pharmaceutically acceptable derivative thereof, for use according to any one of claims 1 to 3, wherein Etifoxine is present in the form of R-Etifoxine, S-Etifoxine, or a mixture thereof.
5. Etifoxine, or a pharmaceutically acceptable derivative thereof, for use according to any one of claims 1 to 4, wherein the treatment comprises oral, parenteral, subcutaneous, intravenous, mucosal, intracutaneous, intramuscular, intraperitoneal, transdermal, intranasal, transmucosal, rectal, sublingual, topical, intravaginal, gastrointestinal, buccal administration, or administration by inhalation, preferably wherein the treatment comprises oral, sublingual, buccal, parenteral, intravenous, subcutaneous, intravaginal, transdermal administration, or administration by inhalation, more preferably wherein the treatment comprises oral, parenteral, topical, transdermal administration, or administration by inhalation.
6. A pharmaceutical composition comprising Etifoxine, or a pharmaceutically acceptable derivative thereof, and a pharmaceutically acceptable excipient for use in the treatment of a disease related to activated mast cells.
7. The pharmaceutical composition according to claim 6, wherein the disease is selected from the group consisting of asthma, allergy, abdominal aortic aneurysms, anaphylaxis, anaemia due to inflammation and/or oxidative stress, angioedema, atherosclerosis, bladder pain syndrome, chronic fatigue syndrome, chronic lyme disease, chronic obstructive pulmonary disease, collagen diseases, deep vein thrombosis, disturbances of the coagulation system, dysautonomia, endometriosis, eosinophilic gastroenteritis, fibromyalgia, food intolerance, functional dyspepsia, Gulf war illness, H1N1 influenza, histamine intolerance, hypercholesterolemia, hypermobility-type Ehlers Danlos syndrome, idiopathic anaphylaxis, inflammatory bowel disease, interstitial cystitis, irritable bowel syndrome, ischemic stroke, hemorrhagic stroke, itch, keloidal scars, systemic mastocytosis, metabolic syndrome, multiple chemical sensitivity, obesity, osteoarthritis, osteoporosis, osteopenia, osteolytic lesions, pemphigus vulgaris, postural orthostatic tachycardia syndrome, post-traumatic headache, pre-term delivery, rosacea, sepsis, sickle cell disease, Sjogren's syndrome, primary Sjogren's syndrome, spondylarthritis, systemic mast cell activation disease, thrombosis, urticaria, vulvodynia, tumor growth, metastasis, cancer, autoimmune disorders, inflammatory disorders, fibrotic disorders, and viral hemorrhagic fever caused by a virus, wherein the virus is a member of at least one virus family selected from the group consisting of Arenaviridae, Filoviridae, Bunyaviridae, Flaviviridae, and Rhabdoviridae, preferably wherein the virus comprises Dengue virus, Ebola virus, Marburg virus, Hanta virus, West Nile virus, Saint Louis encephalitis, Kunjin virus, Lassa virus, Crimean-Congo hemorrhagic fever virus, Rift Valley fever virus, yellow fever virus, Japanese encephalitis virus, Lujo virus, Junin virus, Argentine hemorrhagic fever virus, Brazilian hemorrhagic fever virus, Bolivian hemorrhagic fever virus, Argentine hemorrhagic fever virus, Garissa virus, SFTS virus, Hantan virus, Omsk hemorrhagic fever virus, Kyasanur forest disease virus, Langat virus, Alkhurma virus, or Henipa virus.
8. The pharmaceutical composition according to claim 6 or 7, wherein the disease is selected from the group consisting of allergy, asthma, angioedema, chronic fatigue syndrome, chronic lyme disease, deep vein thrombosis, endometriosis, fibromyalgia, Gulf war illness, inflammatory bowel disease, interstitial cystitis, irritable bowel syndrome, ischemic stroke, hemorrhagic stroke, systemic mastocytosis, postural orthostatic tachycardia syndrome, systemic mast cell activation disease, thrombosis, tumor growth, metastasis, cancer, fibrotic disorders, autoimmune disorders, and viral hemorrhagic fever caused by a virus, wherein the virus comprises Dengue virus, West Nile virus, Crimean-Congo hemorrhagic fever virus, or Japanese encephalitis virus, preferably wherein the disease is selected from the group consisting of allergy, asthma, autoimmune disorders, pulmonary fibrosis, chronic fatigue syndrome, chronic lyme disease, endometriosis, fibromyalgia, Gulf war illness, inflammatory bowel disease, interstitial cystitis, irritable bowel syndrome, systemic mastocytosis, postural orthostatic tachycardia syndrome, post-traumatic headache, systemic mast cell activation disease, fibrotic disorders, tumor growth, metastasis, cancer, and viral hemorrhagic fever caused by a virus, wherein the virus comprises Dengue virus, West Nile virus, Crimean-Congo hemorrhagic fever virus, or Japanese encephalitis virus, more preferably wherein the disease is selected from the group consisting of allergy, asthma, autoimmune disorders, chronic fatigue syndrome, chronic lyme disease, fibromyalgia, fibrotic disorders, inflammatory bowel disease, irritable bowel syndrome, systemic mastocytosis, systemic mast cell activation disease, and viral hemorrhagic fever caused by a virus, wherein the virus comprises Dengue virus, and most preferably wherein the disease is selected from the group consisting of allergy, asthma, fibromyalgia, fibrotic disorders, inflammatory bowel disease, irritable bowel syndrome, systemic mastocytosis, systemic mast cell activation disease, and viral hemorrhagic fever caused by a virus, wherein the virus comprises Dengue virus.
9. The pharmaceutical composition according to any one of claims 6 to 8, wherein the pharmaceutical composition further comprises a compound selected from the group consisting of binder, filler, diluent, disintegrating agent, lubricant, glidant, flavouring agent, wetting agent, emulsifying agent, pH buffering agent, sweetening agent, and a mixture thereof, preferably wherein the pharmaceutical composition further comprises at least one compound selected from the group consisting of starch, glucose, lactose, sucrose, gelatin, malt, rice, malt, flour, silica gel, sodium stearate, glycerol monostearate, talc, sodium chloride, dried skim milk, glycerol, propylene, glycol, and water.
10. The pharmaceutical composition according to any one of claims 6 to 9, wherein the treatment comprises oral, parenteral, subcutaneous, intravenous, mucosal, intracutaneous, intramuscular, intraperitoneal, transdermal, intranasal, transmucosal, rectal, sublingual, topical, intravaginal, gastrointestinal, topical, buccal administration, or administration by inhalation, preferably wherein the treatment comprises oral, sublingual, buccal, parenteral, intravenous, subcutaneous, topical, intravaginal, transdermal administration, or administration by inhalation, more preferably wherein the treatment comprises oral, parenteral, topical, transdermal administration, or administration by inhalation.
11. An in vitro or ex-vivo method of inhibiting mast cell activation, comprising contacting the mast cell with Etifoxine, or a pharmaceutically acceptable derivative thereof.
12. The method according to claim 11, wherein the inhibition of mast cells is associated with a dose-dependent reduction of degranulation, wherein the degranulation comprises release of pre-stored mediators and/or de novo synthesized mediators, preferably wherein the pre-stored mediators and/or the de novo synthesized mediators comprise biogenic amine, proteoglycan, protease, tryptase, lysosomal enzyme, growth factor, phospholipid metabolite, cytokine, chemokine, peptide, or a mixture thereof.
13. The method according to claim 11 or 12, wherein the pre-stored mediators and/or the de novo synthesized mediators comprise biogenic amine comprising histamine, serotonin, or polyamines, proteoglycan comprising heparin, chondroitin sulfates, or serglycin, protease comprising matrix metalloproteinases, chymase-1, cathepsin G, granzyme B, carboxypeptidase A3, or tryptases comprising tryptase-α, tryptase-βI, tryptase-βII, tryptase-βIII, tryptase-γ, and tryptase-δ, lysosomal enzyme comprising β-glucuronidase, β-hexosaminidase, or arylsulfatase, growth factor comprising GM-CSF, VEGF, NGF, or PDGF, phospholipid metabolite comprising LTB₄, LTC₄, PGD₂, PGE₂, or PAF, cytokine comprising bFGF, SCF, interferon α, interferon β, interferon γ, TNF-α, TGF-β, or interleukins comprising IL-1, IL-3, IL-4, IL-5, IL-6, IL-8, IL-9, IL-10, IL-12, IL-13, IL-14, IL-16, IL-17, IL-18, and IL-25, chemokine comprising MIP-1α, MIP-1β, CXCL8, CCL2, CCL5, CCL7, CCL11, or CCL13, peptide comprising CRH (corticotropin-releasing hormone), urocortin comprising urocortin, urocortin-2, urocortin-3, or VIP (vasoactive intestinal peptide), peroxidase, phospholipase, renin, substance P, leptin, angiogenin, nitric oxide, or a mixture thereof, preferably wherein the pre-stored mediators and/or the de novo synthesized mediators comprise histamine, heparin, tryptases comprising tryptase-α, tryptase-βI, tryptase-βII, tryptase-βIII, tryptase-γ, and tryptase-δ, β-hexosaminidase, MIP-1α, MIP-1β, CXCL8, CCL2, CCL5, CCL7, CCL11, CCL13, renin, substance P, CRH, VEGF, TNF-α, IL1-1, IL-6, IL-17, LTC₄, PGD₂, or a mixture thereof, more preferably wherein the pre-stored mediators and/or the de novo synthesized mediators comprise histamine, heparin, tryptases comprising tryptase-α, tryptase-βI, tryptase-βII, tryptase-βIII, tryptase-γ, and tryptase-δ, β-hexosaminidase, MIP-1α, MIP-1β, CXCL8, CCL2, CCL5, CCL7, CCL11, or CCL13, L1-1, IL-6, IL-17, PGD₂, or a mixture thereof.

1: A method of treating a disease related to activated mast cells, the method comprising:
administering etifoxine, or a pharmaceutically acceptable derivative thereof, to a subject in need thereof.

2-10. (canceled)

11: An in vitro or ex-vivo method of inhibiting mast cell activation, comprising contacting the mast cell with Etifoxine, or a pharmaceutically acceptable derivative thereof.

12: The method according to claim 11, wherein the inhibition of mast cells is associated with a dose-dependent reduction of degranulation, wherein the degranulation comprises release of pre-stored mediators and/or de nova synthesized mediators, preferably wherein the pre-stored mediators and/or the de nova synthesized mediators comprise biogenic amine, proteoglycan, protease, tryptase, lysosomal enzyme, growth factor, phospholipid metabolite, cytokine, chemokine, peptide, or a mixture thereof.

13: The method according to claim 12, wherein the pre-stored mediators and/or the de nosy) synthesized mediators comprise biogenic amine comprising histamine, serotonin, or polyamines, proteoglycan comprising heparin, chondroitin sulfates, or serglycin, protease comprising matrix metalloproteinases, chymase-1, cathepsin G, granzyme B, carboxypeptidase A3, or tryptases comprising tryptase-α, tryptase-βI, tryptase-βII, tryptase-γ, and tryptase-δ, lysosomal enzyme comprising β-glucuronidase, β-hexosaminidase, or arylsulfatase, growth factor comprising GM-CSF, VEGF, NGF, or PDGF, phospholipid metabolite comprising LTB₄, LTC₄, PGD₂, PGE₂, or PAF, cytokine comprising bFGF, SCF, interferon α, interferon β, interferon γ, TNF-α, TGF-β, or interleukins comprising IL-1, IL-3, IL-4, IL-5, IL-6, IL-8, IL-9, IL-10, IL-12, IL-13, IL-14, IL-16, IL-17, IL-18, and IL-25, chemokine comprising MIP-1α, MIP-1β, CXCL8, CCL2, CCL5, CCL7, CCL11, or CCL13, peptide comprising CRH (corticotropin-releasing hormone), urocortin comprising urocortin, urocortin-2, urocortin-3, or VIP (vasoactive intestinal peptide), peroxidase, phospholipase, renin, substance P, leptin, angiogenin, nitric oxide, or a mixture thereof, preferably wherein the pre-stored mediators and/or the de novo synthesized mediators comprise histamine, heparin, tryptases comprising tryptase-α, tryptase-βI, tryptase-βII, tryptase-βIII, tryptase-γ, and tryptase-δ, β-hexosaminidase, MIP-1α, MIP-1β, CXCL8, CCL2, CCL5, CCL7, CCL11, CCL13, renin, substance P, CRH, VEGF, TNF-α, IL1-1, IL-6, IL-17, LTC₄, PGD₂, or a mixture thereof, more preferably wherein the pre-stored mediators and/or the de novo synthesized mediators comprise histamine, heparin, tryptases comprising tryptase-α, tryptase-βI, tryptase-βII, tryptase-βIII, tryptase-γ, and tryptase-δ, β-hexosaminidase, MIP-1α, MIP-β1, CXCL8, CCL2, CCL5, CCL7, CCL11, or CCL13, L1-1, IL-6, IL-17, PGD₂, or a mixture thereof.
